(19) United States
(12) Patent Application Publication
(10) Pub. No.: US 2008/0243113 A1
(43) Pub. Date: Oct. 2, 2008

(54) MODIFICATION OF STENT SURFACES TO IMPART FUNCTIONALITY

(76) Inventors: V. Prasad Shastri, Nashville, TN (US); Thomas Soike, Johnson City, TN (US); Chenxia Guan, Nashville, TN (US)

Correspondence Address: Ballard Spahr Andrews & Ingersoll, LLP, SUITE 1000, 999 PEACHTREE STREET, ATLANTA, GA, 30309-3915, US

(21) Appl. No.: 11/937,439

(22) Filed: Nov. 8, 2007

Related U.S. Application Data
(60) Provisional application No. 60/864,923, filed on Nov. 8, 2006.
(60) Provisional application No. 60/865,016, filed on Nov. 9, 2006.

(51) Int. Cl. A61B 18/18; B32B 5/02; B32B 27/08; B05D 1/36; A61F 2/82; A61B 17/58

(52) U.S. Cl. 606/33; 428/515; 428/141; 427/407.1; 623/1.46; 606/76; 623/1.34; 623/1.42

(57) ABSTRACT

In one aspect, the invention relates to coated substrates comprising a substrate having a surface, a cationic polymer layer adjacent the surface of the substrate, an anionic polymer layer adjacent the cationic polymer layer and methods for producing and using same. In one aspect, the cationic polymer layer comprises at least one residue of a first compound having the structure:

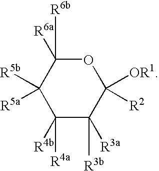

In a further aspect, the anionic polymer layer comprises at least one residue of a compound having the structure:

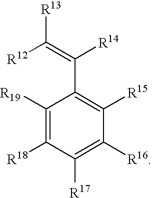

In a yet further aspect, at least one nanoparticle or microparticle is positioned within one or both of the anionic polymer layer and the cationic polymer layer. In a still further aspect, the outermost polymer layer has a surface having fractal characteristics. This abstract is intended as a scanning tool for purposes of searching in the particular art and is not intended to be limiting of the present invention.

### CROSS-REFERENCE TO RELATED APPLICATIONS

This application claims the benefit of U.S. Application Nos. 60/864,923, filed Nov. 8, 2006, and 60/865,016, filed Nov. 9, 2006, which are hereby incorporated herein by reference in their entireties.

### ACKNOWLEDGEMENT OF GOVERNMENT SUPPORT

This work was supported in part by the National Institutes of Health (R24-AI47739-03) and the National Science Foundation REU Program (NSF DMR02-43676). The United States Government may have certain rights in this invention.

### BACKGROUND

Blocked coronary arteries are typically treated by a stenting procedure. This involves dilation of the occluded artery with a metallic or polymer stent, as shown in FIG. 1. A stent in essence is a cylindrical cage that when placed at the site of damage in the artery and expanded using a balloon catheter can serve to mechanically stabilize the walls of the artery and prevent it from collapsing and clogging. Over one million stent procedures are performed annually. In roughly 30% of these cases, the stents fail, and the stent-supported artery constricts. In many instances, early intervention could have yielded more positive outcomes. The ability to extract information concerning the changes occurring in the tissue surrounding a stent can, therefore, facilitate such intervention.

There are, in general, two problems associated with conventional stenting procedures. The first relates to the biological response following stenting that can result in an uncontrolled proliferation of scar tissue from the vessel wall, resulting in a clinical conditions called neo intimal hyperplasia. This uncontrolled growth of scar tissue can in essence block the opened artery. Currently, this is treated by the local delivery of Paclitaxel (Taxol) using the stent as the delivery vehicle, wherein the drug is dispersed in a polymer coating on the stent surface. Such stents are called drug-eluting stents.

Another clinically relevant issue with the polymeric and metallic stents is their relative lucency to X-ray radiation makes it difficult to visualize using common imaging modalities, such as traditional X-ray radiography and CT-Scan. It has been shown that the drug-eluting stents, while effective in the short term, are ineffective at preventing intimal hyperplasia in the long run. So there exists a need to be able to image the vicinity of the stent using traditional imaging modalities such as CT and MRI so that the fate of the tissue adjacent to the stent can be monitored and serve as a predictive element in the diagnosis of recurring intimal hyperplasia.

Thus, conventional processes for stenting typically fail to adequately prevent intimal hyperplasia and to adequately extract information concerning the surrounding tissue. Therefore, there remains a need for methods and compositions that overcome these deficiencies and that effectively provide improved stents and stenting procedures.

### SUMMARY

As embodied and broadly described herein, the invention, in one aspect, relates to coated substrates and methods for producing same.

Disclosed are coated substrates comprising a substrate having a surface, a cationic polymer layer adjacent the surface of the substrate, wherein the cationic polymer layer comprises at least one residue of a first compound having the structure:

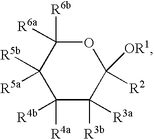

wherein R¹ is hydrogen or alkyl; wherein R², R^{3a}, R^{4a}, R^{4b}, R^{5a}, R^{5b}, R^{6a}, and R^{6b} are, independently, hydrogen, hydroxyl, alkyl, aryl, alkoxy, carboxyl, ester, amino, or amide, with the provisos that at least one of R², R^{3a}, R^{3b}, R^{4a}, R^{4b}, R^{5a}, R^{5b}, R^{6a}, and R^{6b} is amino and that at least one of R², R^{3a}, R^{3b}, R^{4a}, R^{4b}, R^{5a}, R^{5b}, R^{6a}, and R^{6b} is hydroxyl or alkoxy; and an anionic polymer layer adjacent the cationic layer, wherein the anionic polymer layer comprises at least one residue of a compound having the structure:

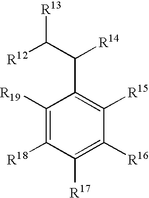

wherein R¹², R¹³, and R¹⁴ are, independently, hydrogen, alkyl, aryl, carboxyl, or ester; and wherein R¹⁵, R¹⁶, R¹⁷, R¹⁸, and R¹⁹ are, independently, hydrogen, alkyl, aryl, alkoxy, amino, amide, carboxyl, or ester, with the proviso that at least one of R¹⁵, R¹⁶, R¹⁷, R¹⁸, and R¹⁹ is SO₃R¹¹, wherein R¹¹ is hydrogen or alkyl.

Also disclosed are coated substrates comprising a substrate having a surface, a cationic polymer layer adjacent the surface of the substrate, an anionic polymer layer adjacent the cationic polymer layer, and at least one nanoparticle or microparticle positioned within the anionic polymer layer.

Also disclosed are methods of making a coated substrate comprising the steps of providing a substrate having a surface; contacting the surface with an ionic polymer solution, thereby disposing an ionic polymer layer adjacent to the surface; and contacting the ionic polymer layer with a counterionic polymer solution, thereby disposing a counterionic polymer layer adjacent to the ionic polymer layer, wherein one of the ionic polymer layer and the counterionic polymer layer comprises at least one residue of a first compound having the structure:

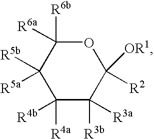

wherein R¹ is hydrogen or alkyl; wherein R², R^{3a}, R^{3b}, R^{4a}, R^{4b}, R^{5a}, R^{5b}, R^{6a}, and R^{6b} are, independently, hydrogen, hydroxyl, alkyl, alkoxy, carboxyl, ester, amino, or amide, with the provisos that at least one of R², R^{3a}, R^{3b}, R^{4a}, R^{4b}, R^{5a}, R^{5b}, R^{6a}, and R^{6b} is amino and that at least one of R², R^{3a}, R^{3b}, R^{4a}, R^{4b}, R^{5a}, R^{5b}, R^{6a}, and R^{6b} is hydroxyl or alkoxy; and wherein the other of the ionic polymer layer and the counterionic polymer layer comprises at least one residue of a compound having the structure:

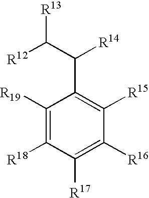

wherein R¹², R¹³, and R¹⁴ are, independently, hydrogen, alkyl, carboxyl, or ester; and wherein R¹⁵, R¹⁶, R¹⁷, R¹⁸, and R¹⁹ are, independently, hydrogen, alkyl, alkoxy, amino, amide, carboxyl, or ester, with the proviso that at least one of R¹⁵, R¹⁶, R¹⁷, R¹⁸, and R¹⁹ is SO₃R¹¹ wherein R¹¹ is hydrogen or alkyl.

Also disclosed are methods of making a coated substrate comprising the steps of providing a substrate having a surface; contacting the surface with an ionic polymer solution, thereby disposing an ionic polymer layer adjacent to the surface; and contacting the ionic polymer layer with a counterionic polymer solution, thereby disposing a counterionic polymer layer adjacent to the ionic polymer layer, wherein one or both of the ionic polymer solution and the counterionic polymer solution further comprises least one nanoparticle or microparticle.

Also disclosed are methods of treating comprising the step of implanting a disclosed coated substrate of or the product produced by a disclosed method into a subject.

Also disclosed are methods of performing radio frequency ablation comprising the steps of providing a disclosed coated substrate or a product produced by a disclosed method, wherein the coated substrate or the product further comprises at least one metal nanoparticle or metal microparticle; and exposing the coated substrate or the product to radio frequency radiation.

Also disclosed are products produced by the disclosed methods.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

### BRIEF DESCRIPTION OF THE FIGURES

The accompanying drawings, which are incorporated in and constitute a part of this specification, illustrate several embodiments and together with the description illustrate the disclosed compositions and methods.

FIG. 1 shows the process of inserting a balloon-expandable stent into a coronary artery. The balloon causes the stent to inflate, while cracking and compressing the plaque found in the artery as well. The balloon is then deflated and withdrawn with the catheter while the stent remains.

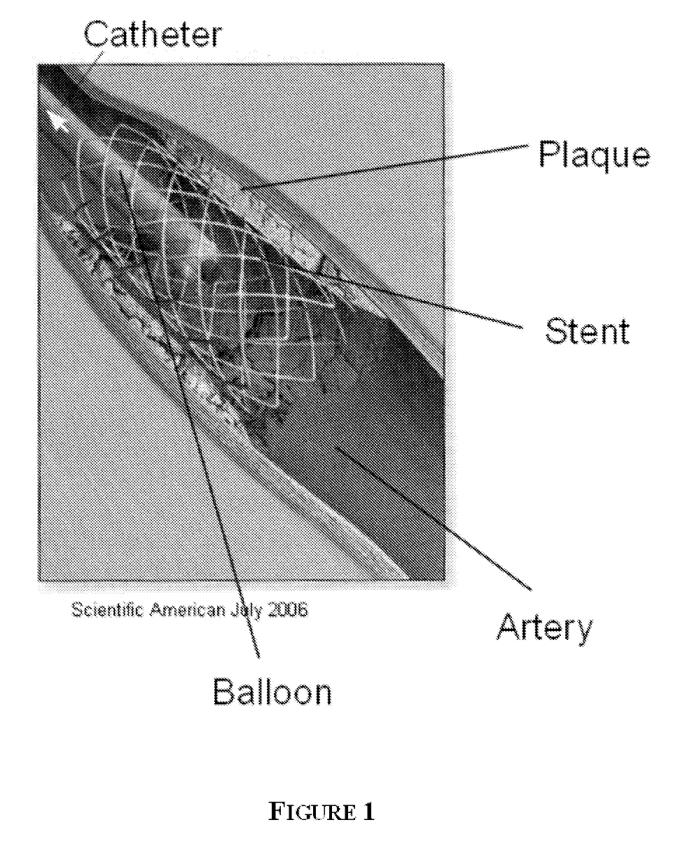

FIG. 2 shows micrographs of coated substrates with surface coverage as a function of number of layers.

FIG. 3 shows a graph illustrating percentage of nanoparticles as a function of number of layers at different magnifications.

FIG. 4 shows micrographs of coated substrates with percentage of nanoparticles as a function of nanoparticle concentration.

FIG. 5 shows a graph illustrating percentage of nanoparticles as a function of nanoparticle concentration.

FIG. 6 shows micrographs of coated substrates with percentage of nanoparticles as a function of dipping time.

FIG. 7 shows a graph illustrating percentage of nanoparticles as a function of dipping time.

FIG. 8 shows a schematic of a general procedure for preparing coated substrates.

FIG. 9 shows a micrograph of the coating produced in Example 1.

FIG. 10 shows a micrograph of the coating produced in Example 2.

FIG. 11 shows a micrograph of the coating produced in Example 3.

FIG. 12 shows a micrograph of the coating produced in Example 4.

FIG. 13 shows a micrograph of the coating produced in Example 5.

FIG. 14 shows a micrograph of the coating produced in Example 6.

FIG. 15 shows a micrograph of the coating produced in Example 7.

FIG. 16 shows a micrograph of the coating produced in Example 8.

FIG. 17 shows a micrograph of the coating produced in Example 9.

FIG. 18 shows a micrograph of the coating produced in Example 10.

FIG. 19 shows a micrograph of the coating produced in Example 11.

FIG. 20 shows a micrograph of the coating produced in Example 12.

FIG. 21 shows a micrograph of the coating produced in Example 13.

FIG. 22 shows a micrograph of the coating produced in Example 14.

FIG. 23 shows a micrograph of the coating produced in Example 15.

FIG. 24 shows micrographs of the coating produced in Example 16.

FIG. 25 shows PS-NP surface coverage. 20 minute dipping times and 0.4% nanoparticle solutions produced the most surface coverage.

FIG. 26 shows an SEM image of a surface modified with gold nanoshells (10⁴×).

FIG. 27 shows an SEM image of a surface modified with PS-NP 5000×.

FIG. 28 shows a schematic of the preparation and composition of exemplary coated substrates.

FIG. 29 shows micro-CT images of exemplary coated substrates from FIG. 28.

FIG. 30 shows a release profile of fluorescein diacetate from a stainless steel foil surface modified with poly(lactic-co-glycolic) acid nanoparticles containing fluorescein diacetate.

FIG. 31 shows release profiles of bovine serum albumin (BSA) from stainless steel foil surfaces modified with a poly(styrene sulfonate) functionalized poly(lactic-co-glycolic) acid nanoparticle containing bovine serum albumin.

FIG. 32 shows release profiles of horseradish peroxidase (HRP) from stainless steel foil surfaces modified with a poly(styrene sulfonate) functionalized poly(lactic-co-glycolic) acid nanoparticles containing HRP.

FIG. 33 shows: a) the influence of acetone volume fraction on nanoparticle size; and b) nanoparticle size as a function of increasing glycerol volume fraction (viscosity).

FIG. 34 shows: a) changes in the zeta potential of functionalized nanoparticles as a function of pH; and b) scanning electron micrographs of PLGA modified with (A) poly(acrylic acid) and (B) poly(styrene sulfonate).

FIG. 35 shows XPS C 1s spectra of: a) unmodified PLGA nanoparticles, and b) poly(ethylene oxide) modified PLGA nanoparticles.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DESCRIPTION

The present invention can be understood more readily by reference to the following detailed description of aspects of the invention and the Examples included therein.

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can need to be independently confirmed.

### A. DEFINITIONS

As used in the specification and the appended claims, the singular forms “a,” “an” and “the” include plural referents unless the context clearly dictates otherwise. Thus, for example, reference to “a component,” “a polymer,” or “a residue” includes mixtures of two or more such components, polymers, or residues, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the terms “optional” or “optionally” means that the subsequently described event or circumstance may or may not occur, and that the description includes instances where said event or circumstance occurs and instances where it does not.

As used herein, the term “polymer” refers to a relatively high molecular weight organic compound, natural or synthetic, whose structure can be represented by a repeated small unit, the monomer (e.g., polyethylene, rubber, cellulose). Synthetic polymers are typically formed by addition or condensation polymerization of monomers.

As used herein, the term “copolymer” refers to a polymer formed from two or more different repeating units (monomer residues). By way of example and without limitation, a copolymer can be an alternating copolymer, a random copolymer, a block copolymer, or a graft copolymer. It is also contemplated that, in certain aspects, various block segments of a block copolymer can themselves comprise copolymers.

As used herein, the term “oligomer” refers to a relatively low molecular weight polymer in which the number of repeating units is between two and ten, for example, from two to eight, from two to six, or form two to four. In one aspect, a collection of oligomers can have an average number of repeating units of from about two to about ten, for example, from about two to about eight, from about two to about six, or form about two to about four.

As used herein, the term “cationic” refers to a material having a net positive charge at the pH of adsorption and, preferably, at or about physiological pH of 6.8-7.4 (e.g., 7.2). In one aspect, a layer can be referred to as “cationic” if the layer exhibits a net positive charge during deposition. In a further aspect, a layer can be referred to as “cationic” if the layer exhibits a net positive charge in a coating at or about physiological pH of 6.8-7.4 (e.g., 7.2). As used herein, the term “pH of adsorption” refers to the pH of the material or the solution comprising the material during the step of disposing a layer. Examples of cationic polymers include chitosan, poly(L-lysine), poly(allylamines), polyimidazole, poly(histidines), poly(aspartamine), polylPAM, and poly(N,N-dimethyl isopropylacrylamide.

As used herein, the term “anionic” refers to a material having a net negative charge at the pH of adsorption and, preferably, at or about physiological pH of 6.8-7.4 (e.g., 7.2). In one aspect, a layer can be referred to as “anionic” if the layer exhibits a net negative charge during deposition. In a further aspect, a layer can be referred to as “anionic” if the layer exhibits a net negative charge in a coating at or about physiological pH of 6.8-7.4 (e.g., 7.2). As used herein, the term “pH of adsorption” refers to the pH of the material or the solution comprising the material during the step of disposing a layer. Examples of anionic polymers include poly(styrene sulfonate), hyaluronic acid, alginate, poly(glutamic acid), poly(aspartic acid), poly(acrylic acid), and alginic acid (alginate).

As used herein, the term “polymer layer,” which can also be referred to as “polymer coating,” refers to a thickness of polymeric material. In one aspect, the polymer layer comprises a layer of nanoparticulate material. That is, in one aspect, the polymer layer can comprise polymeric nanoparticles. It is understood that the layer need not be a confluent sheet of material, but can instead comprise a layer of nanoparticles covering at least a portion of a substrate or at least a portion of a further polymer layer. In one aspect, one or more polymer layers can have “fractal characteristics,” as defined herein.

As used herein, the term “effective amount” refers to an amount that is sufficient to achieve a desired result or to have an effect on undesired symptoms, but is generally insufficient to cause adverse side affects. The specific effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In a further aspect, a preparation can be administered in a “diagnostically effective amount”; that is, an amount effective for diagnosis of a disease or condition. In a further aspect, a preparation can be administered in a “therapeutically effective amount”; that is, an amount effective for treatment of a disease or condition. In a further aspect, a preparation can be administered in a “prophylactically effective amount”; that is, an amount effective for prevention of a disease or condition.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, and parenteral administration, including injectable such as intravenous administration, intra-arterial administration, intramuscular administration, and subcutaneous administration. In particular, “administration” can refer to bolus injection with a syringe and needle, or to infusion through a catheter in place within a vessel. A vessel can be an artery or a vein. Administration can be continuous or intermittent. In one aspect, systemic delivery of payloads by transdermal administration into subcutaneous circulation using the solid lipid nanoparticles of the invention can be accomplished in combination with a chemical penetration enhancer. In various aspects, a preparation can be administered therapeutically; that is, administered to treat an existing disease or condition. In further various aspects, a preparation can be administered prophylactically; that is, administered for prevention of a disease or condition. In a further aspect, “administering” and “administration” can refer to administration to cells that have been removed from a subject (e.g., human or animal), followed by re-administration of the cells to the same, or a different, subject.

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

As used herein, the terms “implant,” “implanting,” and “implantation” refer to positioning a substrate within a subject. The positioning can be by way of surgical procedure. For example, implanting can refer to positioning a stent within a vessel (e.g., coronary artery) of a subject by way of endoscopic surgery using a catheter.

As used herein, the term “subject” means any target of administration. The subject can be an animal, for example, a mammal (e.g., a human, horse, pig, rabbit, dog, sheep, goat, non-human primate, cow, cat, guinea pig, or rodent), a fish, a bird or a reptile or an amphibian. The term does not denote a particular age or sex. Thus, adult and newborn subjects, as well as fetuses, whether male or female, are intended to be covered. In a further example, the subject can be a human and can be a patient. A “patient” refers to a subject afflicted with a disease or disorder. In one aspect, a patient can be diagnosed with a need for treatment for a disease or disorder. The term “patient” includes human and veterinary subjects.

As used herein, the terms “biologically active agent” and “bioactive agent” refer to an agent that is capable of providing a local or systemic biological, physiological, or therapeutic effect in the biological system to which it is applied. For example, the bioactive agent can act to control infection or inflammation, enhance cell growth and tissue regeneration, control tumor growth, act as an analgesic, promote anti-cell attachment, and enhance bone growth, among other functions. Other suitable bioactive agents can include anti-viral agents, hormones, antibodies, or therapeutic proteins. Other bioactive agents include prodrugs, which are agents that are not biologically active when administered but, upon administration to a subject are converted to bioactive agents through metabolism or some other mechanism. Examples of biologically active agents that can be used in connection with the invention include, without limitation, one or more of biotin, streptavidin, protein A, protein G, an antibody, antibody fragment F(ab)₂, antibody fragment F(ab)′, a receptor ligand such as VEGF, VLA-4, or TNF-alpha, a neurotransmitter such as serotonin, a receptor antagonist such as muscimol (GABA antagonist), or an antioxidants such as Vitamin E (alpha-tocopherols) or C (ascorbic acid). Additionally, any of the compositions of the invention can contain combinations of two or more bioactive agents.

As used herein, the term “pharmaceutically active agent” refers a “drug” or a “vaccine” and means a molecule, group of molecules, complex or substance administered to an organism for diagnostic, therapeutic, preventative medical, or veterinary purposes. This term include externally and internally administered topical, localized and systemic human and animal pharmaceuticals, treatments, remedies, nutraceuticals, cosmeceuticals, biologicals, devices, diagnostics and contraceptives, including preparations useful in clinical and veterinary screening, prevention, prophylaxis, healing, wellness, detection, imaging, diagnosis, therapy, surgery, monitoring, cosmetics, prosthetics, forensics and the like. This term may also be used in reference to agriceutical, workplace, military, industrial and environmental therapeutics or remedies comprising selected molecules or selected nucleic acid sequences capable of recognizing cellular receptors, membrane receptors, hormone receptors, therapeutic receptors, microbes, viruses or selected targets comprising or capable of contacting plants, animals and/or humans. This term can also specifically include nucleic acids and compounds comprising nucleic acids that produce a bioactive effect, for example deoxyribonucleic acid (DNA) or ribonucleic acid (RNA). Pharmaceutically active agents include the herein disclosed categories and specific examples. It is not intended that the category be limited by the specific examples. Those of ordinary skill in the art will recognize also numerous other compounds that fall within the categories and that are useful according to the invention. Examples include a radiosensitizer, the combination of a radiosensitizer and a chemotherapeutic, a steroid, a xanthine, a beta-2-agonist bronchodilator, an anti-inflammatory agent, an analgesic agent, a calcium antagonist, an angiotensin-converting enzyme inhibitors, a beta-blocker, a centrally active alpha-agonist, an alpha-1-antagonist, an anticholinergic/antispasmodic agent, a vasopressin analogue, an antiarrhythmic agent, an antiparkinsonian agent, an antiangina/antihypertensive agent, an anticoagulant agent, an antiplatelet agent, a sedative, an ansiolytic agent, a peptidic agent, a biopolymeric agent, an antineoplastic agent, a laxative, an antidiarrheal agent, an antimicrobial agent, an antifungal agent, a vaccine, a protein, or a nucleic acid. In a further aspect, the pharmaceutically active agent can be coumarin, albumin, steroids such as betamethasone, dexamethasone, methylprednisolone, prednisolone, prednisone, triamcinolone, budesonide, hydrocortisone, and pharmaceutically acceptable hydrocortisone derivatives; xanthines such as theophylline and doxophylline; beta-2-agonist bronchodilators such as salbutamol, fenterol, clenbuterol, bambuterol, salmeterol, fenoterol; antiinflammatory agents, including antiasthmatic anti-inflammatory agents, antiarthritis antiinflammatory agents, and non-steroidal antiinflammatory agents, examples of which include but are not limited to sulfides, mesalamine, budesonide, salazopyrin, diclofenac, pharmaceutically acceptable diclofenac salts, nimesulide, naproxene, acetominophen, ibuprofen, ketoprofen and piroxicam; analgesic agents such as salicylates; calcium channel blockers such as nifedipine, amlodipine, and nicardipine; angiotensin-converting enzyme inhibitors such as captopril, benazepril hydrochloride, fosinopril sodium, trandolapril, ramipril, lisinopril, enalapril, quinapril hydrochloride, and moexipril hydrochloride; beta-blockers (i.e., beta adrenergic blocking agents) such as sotalol hydrochloride, timolol maleate, esmolol hydrochloride, carteolol, propanolol hydrochloride, betaxolol hydrochloride, penbutolol sulfate, metoprolol tartrate, metoprolol succinate, acebutolol hydrochloride, atenolol, pindolol, and bisoprolol fumarate; centrally active alpha-2-agonists such as clonidine; alpha-1-antagonists such as doxazosin and prazosin; anticholinergic/antispasmodic agents such as dicyclomine hydrochloride, scopolamine hydrobromide, glycopyrrolate, clidinium bromide, flavoxate, and oxybutynin; vasopressin analogues such as vasopressin and desmopressin; antiarrhythmic agents such as quinidine, lidocaine, tocainide hydrochloride, mexiletine hydrochloride, digoxin, verapamil hydrochloride, propafenone hydrochloride, flecainide acetate, procainamide hydrochloride, moricizine hydrochloride, and disopyramide phosphate; antiparkinsonian agents, such as dopamine, L-Dopa/Carbidopa, selegiline, dihydroergocryptine, pergolide, lisuride, apomorphine, and bromocryptine; antiangina agents and antihypertensive agents such as isosorbide mononitrate, isosorbide dinitrate, propranolol, atenolol and verapamil; anticoagulant and antiplatelet agents such as coumadin, warfarin, acetylsalicylic acid, and ticlopidine; sedatives such as benzodiazapines and barbiturates; ansiolytic agents such as lorazepam, bromazepam, and diazepam; peptidic and biopolymeric agents such as calcitonin, leuprolide and other LHRH agonists, hirudin, cyclosporin, insulin, somatostatin, protirelin, interferon, desmopressin, somatotropin, thymopentin, pidotimod, erythropoietin, interleukins, melatonin, granulocyte/macrophage-CSF, and heparin; antineoplastic agents such as etoposide, etoposide phosphate, cyclophosphamide, methotrexate, 5-fluorouracil, vincristine, doxorubicin, cisplatin, hydroxyurea, leucovorin calcium, tamoxifen, flutamide, asparaginase, altretamine, mitotane, and procarbazine hydrochloride; laxatives such as senna concentrate, casanthranol, bisacodyl, and sodium picosulphate; antidiarrheal agents such as difenoxine hydrochloride, loperamide hydrochloride, furazolidone, diphenoxylate hdyrochloride, and microorganisms; vaccines such as bacterial and viral vaccines; antimicrobial agents such as penicillins, cephalosporins, and macrolides, antifungal agents such as imidazolic and triazolic derivatives; and nucleic acids such as DNA sequences encoding for biological proteins, and antisense oligonucleotides.

As used herein, the term “magnetically active agent” refers to a material that responds to a magnetic field or is capable of exerting an attractive or repulsive force on other magnetic materials. A magnetically active agent can bear various functionalities and can exhibit a net positive or negative charge at physiological pH. In one aspect, a magnetically active agent can be provided in nanoparticular form or in microparticular form. A magnetically active agent can be, for example, a diamagnetic, paramagnetic, ferromagnetic, and/or ferromagnetic material. Examples of magnetically active agents include particles or clusters of Magnetite, Maghemite, Jacobsite, Trevorite, Magnesioferrite, Pyrrhotite, Greigite, Feroxyhyte, Iron, Nickel, Cobalt, Awaruite, Wairauite, Manganese salts, or mixtures thereof.

As used herein, the term “imaging agent” refers to any substance useful for imaging applications, as known to those of skill in the art. Examples of imaging agents include radioconjugate, cytotoxin, cytokine, Gadolinium-DTPA or a quantum dot, iron oxide, manganese oxide. An imaging agent can bear various functionalities and can exhibit a net positive or negative charge at physiological pH. In one aspect, an imaging agent can be provided in nanoparticular form or in microparticular form. In a further aspect, an imaging agent comprises Gadolinium-DTPA and iron oxide nanoparticles (magnetite), as specific MRI contrast agents. In a yet further aspect, an imaging agent comprises at least one near infrared dye, for example near infrared dyes based on a porphyrin and/or a phthalocyanine. See Ghoroghchian et al., Near-infrared-emissive polymersomes: Self-assembled soft matter for in vivo optical imaging, PNAS, 2005, vol. 102, no. 8, 2922-2927. In a still further aspect, the imaging agent comprises two or more quantum dots, wherein the two or more quantum dots have different emission wavelengths. It is understood more than one imaging agent can be used in connection with the disclosed inventions, such as quantum dot—Gd-DTPA-iron oxide nanoparticle co-encapsulated species.

As used herein, the term “fractal characteristics” refers to geometry as described by Mandelbrot's definition of fractal geometry; that is containing fractions of dimensions. Examples of such geometry include fern-like morphology or hyper-branched structures. A surface having fractal characteristics can have a very large surface area, as compared to non-fractal surfaces. For example, a surface having fractal characteristics can have greater than about 100%, greater than about 125%, greater than about 150%, greater than about 200%, greater than about 300%, greater than about 400%, greater than about 500%, greater than about 1,000%, greater than about 10,000% of the surface area of a comparable non-fractal surface. An interface between two surfaces that has fractal characteristics can have a very intimate contact between the two surfaces, due at least in part to the fractal characteristics of one or both surfaces. In one aspect, the disclosed coatings can exhibit fractal characteristics. That is, the surface coatings can have a fractal morphology. In a further aspect, a disclosed anionic polymer layer can yield a fractal structure. In a yet further aspect, an outermost polymer layer of the disclosed coated substrates can have a surface having fractal characteristics.

A residue of a chemical species, as used in the specification and concluding claims, refers to the moiety that is the resulting product of the chemical species in a particular reaction scheme or subsequent formulation or chemical product, regardless of whether the moiety is actually obtained from the chemical species. Thus, an ethylene glycol residue in a polyester refers to one or more —OCH₂CH₂O— units in the polyester, regardless of whether ethylene glycol was used to prepare the polyester. Similarly, a sebacic acid residue in a polyester refers to one or more —CO(CH₂)₈CO— moieties in the polyester, regardless of whether the residue is obtained by reacting sebacic acid or an ester thereof to obtain the polyester.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc.

In defining various terms, “A¹,” “A²,” “A³,” and “A⁴” are used herein as generic symbols to represent various specific substituents. These symbols can be any substituent, not limited to those disclosed herein, and when they are defined to be certain substituents in one instance, they can, in another instance, be defined as some other substituents.

The term “alkyl” as used herein is a branched or unbranched saturated hydrocarbon group of 1 to 24 carbon atoms, for example 1 to 12 carbon atoms or 1 to 6 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can also be substituted or unsubstituted. The alkyl group can be substituted with one or more groups including, but not limited to, substituted or unsubstituted alkyl, cycloalkyl, alkoxy, amino, ether, halide, hydroxy, nitro, silyl, sulfo-oxo, or thiol, as described herein. A “lower alkyl” group is an alkyl group containing from one to six carbon atoms.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” specifically refers to an alkyl group that is substituted with one or more halide, e.g., fluorine, chlorine, bromine, or iodine. The term “alkoxyalkyl” specifically refers to an alkyl group that is substituted with one or more alkoxy groups, as described below. The term “alkylamino” specifically refers to an alkyl group that is substituted with one or more amino groups, as described below, and the like. When “alkyl” is used in one instance and a specific term such as “alkylalcohol” is used in another, it is not meant to imply that the term “alkyl” does not also refer to specific terms such as “alkylalcohol” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, norbornyl, and the like. The term “heterocycloalkyl” is a type of cycloalkyl group as defined above, and is included within the meaning of the term “cycloalkyl,” where at least one of the carbon atoms of the ring is replaced with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, substituted or unsubstituted alkyl, cycloalkyl, alkoxy, amino, ether, halide, hydroxy, nitro, silyl, sulfo-oxo, or thiol as described herein.

The term “polyalkylene group” as used herein is a group having two or more CH₂ groups linked to one another. The polyalkylene group can be represented by the formula —(CH₂)ₐ—, where “a” is an integer of from 2 to 500.

The terms “alkoxy” and “alkoxyl” as used herein to refer to an alkyl or cycloalkyl group bonded through an ether linkage; that is, an “alkoxy” group can be defined as —OA where A¹ is alkyl or cycloalkyl as defined above. “Alkoxy” also includes polymers of alkoxy groups as just described; that is, an alkoxy can be a polyether such as —OA¹-OA² or —OA¹-(OA²)ₐ-OA³, where “a” is an integer of from 1 to 200 and A¹, A², and A³ are alkyl and/or cycloalkyl groups.

The term “alkenyl” as used herein is a hydrocarbon group of from 2 to 24 carbon atoms with a structural formula containing at least one carbon-carbon double bond. Asymmetric structures such as (A¹A²)C═C(A³A⁴) are intended to include both the E and Z isomers. This can be presumed in structural formulae herein wherein an asymmetric alkene is present, or it can be explicitly indicated by the bond symbol C═C. The alkenyl group can be substituted with one or more groups including, but not limited to, substituted or unsubstituted alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkenyl” as used herein is a non-aromatic carbon-based ring composed of at least three carbon atoms and containing at least one carbon-carbon double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, norbornenyl, and the like. The term “heterocycloalkenyl” is a type of cycloalkenyl group as defined above, and is included within the meaning of the term “cycloalkenyl,” where at least one of the carbon atoms of the ring is replaced with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, substituted or unsubstituted alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “alkynyl” as used herein is a hydrocarbon group of 2 to 24 carbon atoms with a structural formula containing at least one carbon-carbon triple bond. The alkynyl group can be unsubstituted or substituted with one or more groups including, but not limited to, substituted or unsubstituted alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol, as described herein.

The term “cycloalkynyl” as used herein is a non-aromatic carbon-based ring composed of at least seven carbon atoms and containing at least one carbon-carbon triple bound. Examples of cycloalkynyl groups include, but are not limited to, cycloheptynyl, cyclooctynyl, cyclononynyl, and the like. The term “heterocycloalkynyl” is a type of cycloalkenyl group as defined above, and is included within the meaning of the term “cycloalkynyl,” where at least one of the carbon atoms of the ring is replaced with a heteroatom such as, but not limited to, nitrogen, oxygen, sulfur, or phosphorus. The cycloalkynyl group and heterocycloalkynyl group can be substituted or unsubstituted. The cycloalkynyl group and heterocycloalkynyl group can be substituted with one or more groups including, but not limited to, substituted or unsubstituted alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein.

The term “aryl” as used herein is a group that contains any carbon-based aromatic group including, but not limited to, benzene, naphthalene, phenyl, biphenyl, phenoxybenzene, and the like. The term “aryl” also includes “heteroaryl,” which is defined as a group that contains an aromatic group that has at least one heteroatom incorporated within the ring of the aromatic group. Examples of heteroatoms include, but are not limited to, nitrogen, oxygen, sulfur, and phosphorus. Likewise, the term “non-heteroaryl,” which is also included in the term “aryl,” defines a group that contains an aromatic group that does not contain a heteroatom. The aryl group can be substituted or unsubstituted. The aryl group can be substituted with one or more groups including, but not limited to, substituted or unsubstituted alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, or thiol as described herein. The term “biaryl” is a specific type of aryl group and is included in the definition of “aryl.” Biaryl refers to two aryl groups that are bound together via a fused ring structure, as in naphthalene, or are attached via one or more carbon-carbon bonds, as in biphenyl.

The term “aldehyde” as used herein is represented by the formula —C(O)H. Throughout this specification “C(O)” is a short hand notation for a carbonyl group, i.e., C═O.

The terms “amine” or “amino” as used herein are represented by the formula NA¹A²A³, where A¹, A², and A³ can be, independently, hydrogen or substituted or unsubstituted alkyl, cycloalkyl, alkenyl, cycloalkenyl, alkynyl, cycloalkynyl, aryl, or heteroaryl group as described herein.

The term “carboxylic acid” as used herein is represented by the formula —C(O)OH.

The term “ester” as used herein is represented by the formula —OC(O)A¹ or —C(O)OA¹, where A¹ can be a substituted or unsubstituted alkyl, cycloalkyl, alkenyl, cycloalkenyl, alkynyl, cycloalkynyl, aryl, or heteroaryl group as described herein. The term “polyester” as used herein is represented by the formula -(A¹O(O)C-A²-C(O)O)ₐ— or -(A¹O(O)C-A²-OC(O))ₐ—, where A¹ and A² can be, independently, a substituted or unsubstituted alkyl, cycloalkyl, alkenyl, cycloalkenyl, alkynyl, cycloalkynyl, aryl, or heteroaryl group described herein and “a” is an integer from 1 to 500. “Polyester” is the term used to describe a group that is produced, for example, by the reaction between a compound having at least two carboxylic acid groups with a compound having at least two hydroxyl groups.

The term “ether” as used herein is represented by the formula A¹OA², where A¹ and A² can be, independently, a substituted or unsubstituted alkyl, cycloalkyl, alkenyl, cycloalkenyl, alkynyl, cycloalkynyl, aryl, or heteroaryl group described herein. The term “polyether” as used herein is represented by the formula -(A¹O-A²O)ₐ—, where A¹ and A² can be, independently, a substituted or unsubstituted alkyl, cycloalkyl, alkenyl, cycloalkenyl, alkynyl, cycloalkynyl, aryl, or heteroaryl group described herein and “a” is an integer of from 1 to 500. Examples of polyether groups include polyethylene oxide, polypropylene oxide, and polybutylene oxide.

The term “halide” as used herein refers to the halogens fluorine, chlorine, bromine, and iodine.

The term “hydroxyl” as used herein is represented by the formula —OH.

The term “ketone” as used herein is represented by the formula A¹C(O)A², where A¹ and A² can be, independently, a substituted or unsubstituted alkyl, cycloalkyl, alkenyl, cycloalkenyl, alkynyl, cycloalkynyl, aryl, or heteroaryl group as described herein.

The term “azide” as used herein is represented by the formula —N₃.

The term “nitro” as used herein is represented by the formula —NO₂.

The term “nitrile” as used herein is represented by the formula —CN.

The term “silyl” as used herein is represented by the formula —SiA¹A²A³, where A¹, A², and A³ can be, independently, hydrogen or a substituted or unsubstituted alkyl, cycloalkyl, alkoxy, alkenyl, cycloalkenyl, alkynyl, cycloalkynyl, aryl, or heteroaryl group as described herein.

The term “sulfo-oxo” as used herein is represented by the formulas —S(O)A¹, —S(O)₂A¹, —OS(O)₂A¹, or —OS(O)₂OA¹, where A¹ can be hydrogen or a substituted or unsubstituted alkyl, cycloalkyl, alkenyl, cycloalkenyl, alkynyl, cycloalkynyl, aryl, or heteroaryl group as described herein. Throughout this specification “S(O)” is a short hand notation for S═O. The term “sulfonyl” is used herein to refer to the sulfo-oxo group represented by the formula —S(O)₂A¹, where A¹ can be hydrogen or a substituted or unsubstituted alkyl, cycloalkyl, alkenyl, cycloalkenyl, alkynyl, cycloalkynyl, aryl, or heteroaryl group as described herein. The term “sulfone” as used herein is represented by the formula A¹S(O)₂A², where A¹ and A² can be, independently, a substituted or unsubstituted alkyl, cycloalkyl, alkenyl, cycloalkenyl, alkynyl, cycloalkynyl, aryl, or heteroaryl group as described herein. The term “sulfoxide” as used herein is represented by the formula A¹S(O)A², where A¹ and A² can be, independently, a substituted or unsubstituted alkyl, cycloalkyl, alkenyl, cycloalkenyl, alkynyl, cycloalkynyl, aryl, or heteroaryl group as described herein.

The term “thiol” as used herein is represented by the formula —SH.

Compounds described herein can contain one or more double bonds and, thus, potentially give rise to cis/trans (E/Z) isomers, as well as other conformational isomers. Unless stated to the contrary, the invention includes all such possible isomers, as well as mixtures of such isomers.

Unless stated to the contrary, a formula with chemical bonds shown only as solid lines and not as wedges or dashed lines contemplates each possible isomer, e.g., each enantiomer and diastereomer, and a mixture of isomers, such as a racemic or scalemic mixture. Compounds described herein can contain one or more asymmetric centers and, thus, potentially give rise to diastereomers and optical isomers. Unless stated to the contrary, the present invention includes all such possible diastereomers as well as their racemic mixtures, their substantially pure resolved enantiomers, all possible geometric isomers, and pharmaceutically acceptable salts thereof. Mixtures of stereoisomers, as well as isolated specific stereoisomers, are also included. During the course of the synthetic procedures used to prepare such compounds, or in using racemization or epimerization procedures known to those skilled in the art, the products of such procedures can be a mixture of stereoisomers.

Disclosed are the components to be used to prepare the compositions of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C—F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

It is understood that the compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

### B. Modification of Stent Surfaces

In contrast to conventional stenting materials, the disclosed invention enables the incorporation of, for example, metallic particles such as gold nanoshells and polymeric and silica nanoparticles, hence rendering the surface amenable to imaging through additional imaging modalities, such as CT and MRI. Furthermore, these nano- and micro-particles can serve as a means of incorporating various drugs that are now released through a controlled release mechanism that is independent of the polymeric coating characteristics. In conventional stent coatings, the physical entrapment of drugs in a polymer coating either through simple solution based coating as in the case of drug-eluting stents or via entrapment in polymers layers in the LBL approach, results in poor control over drug release characteristics as the barrier to diffusion is of limited thickness and cannot be varied significantly without affecting coating characteristics. In contrast, as disclosed herein, the drug delivery component can be independently tuned of the coating characteristics through appropriate choice of the polymer layers (e.g., a cyclic sugar such as cyclodextrin) and/or the composition, and surface area fraction of the nano- or microparticles.

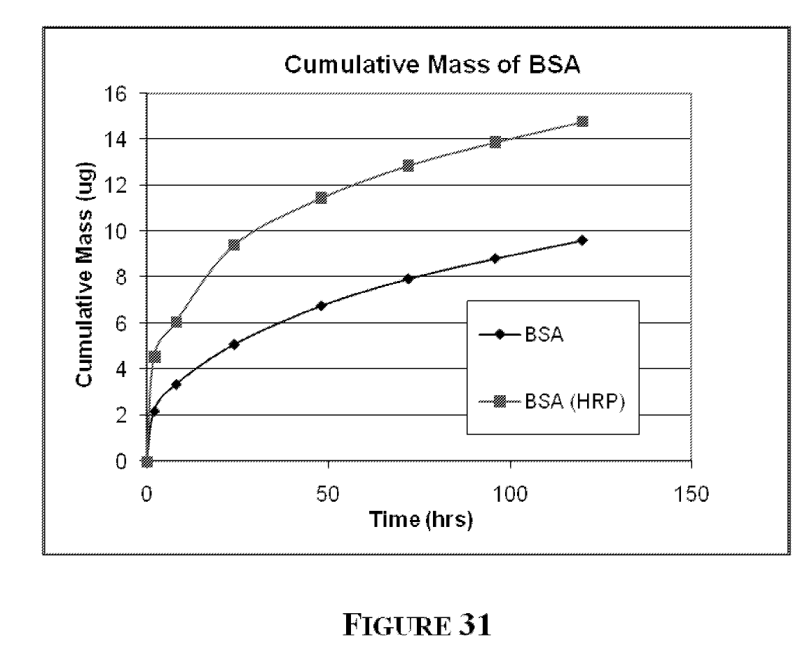

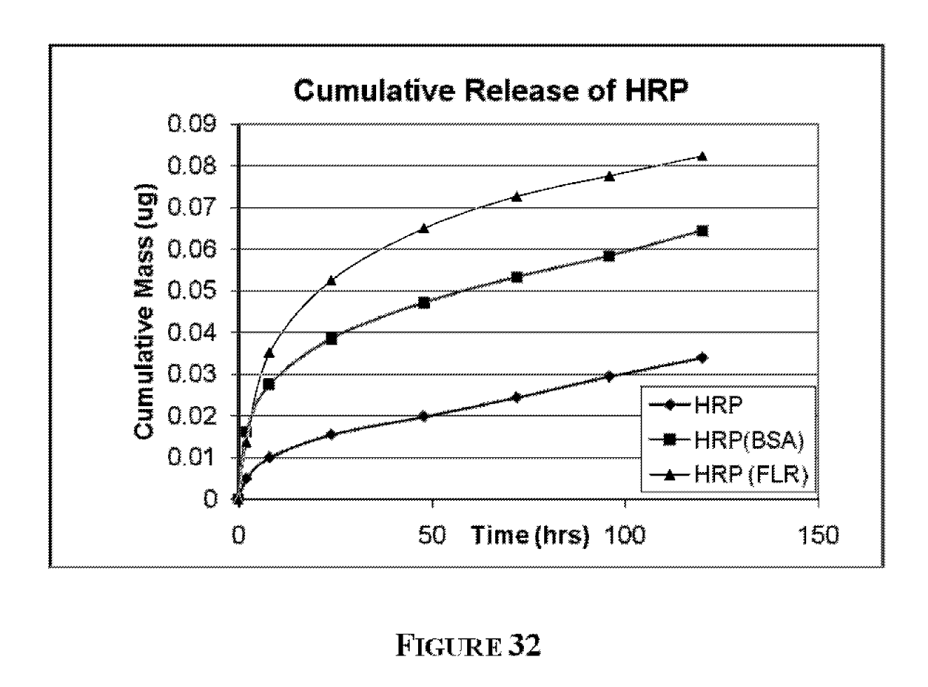

By using 316-L stainless steel, a common alloy used in the fabrication of endovascular and biliary stents, it has been demonstrated that both polymeric and gold nanoparticles (about 30 nm) can be entrapped on the metal surface in a reproducible manner. Furthermore, the surface coverage of the nanoparticles can be altered by varying the number of polymer layers, or NP concentration in solution or the dipping time. Consequently, the stent surfaces can be studied using X-ray and CT modalities. Imaging of the stent and the immediate tissue environment can be accomplished using multiple modalities and, consequently, analysis using co-registration of images from the different modalities (see, e.g., FIGS. 28 and 29). The disclosed invention can be easily applied to cobalt-chromium alloys, titanium, Nitinol, ceramics and polymeric surfaces.

The disclosed coatings and disclosed methods were evaluated as to the relationship between surface coverage and number of layers, particle concentration, and exposure (i.e., dipping) time.

### 1. Surface coverage of nanoparticles as a function of number of Layers

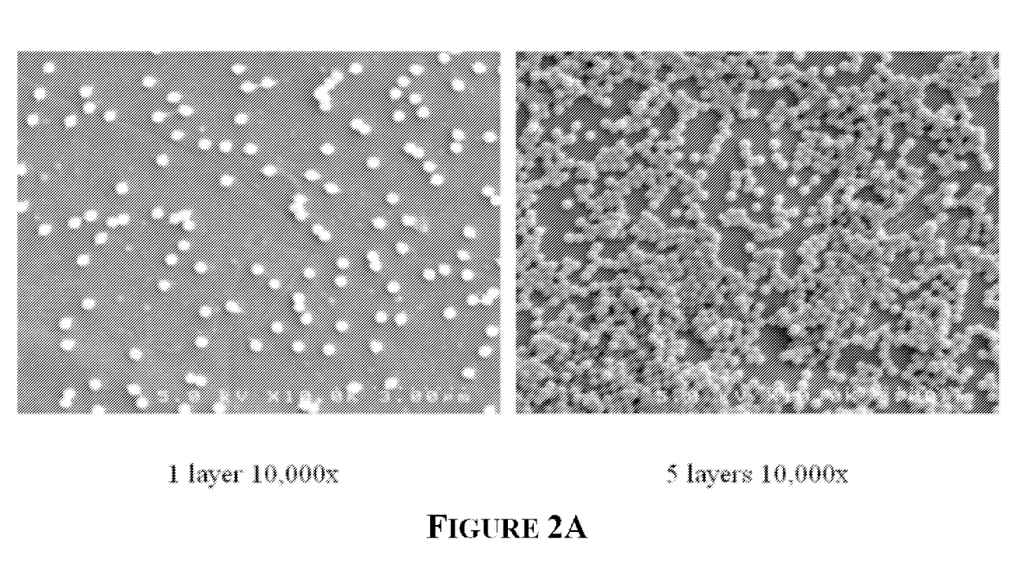

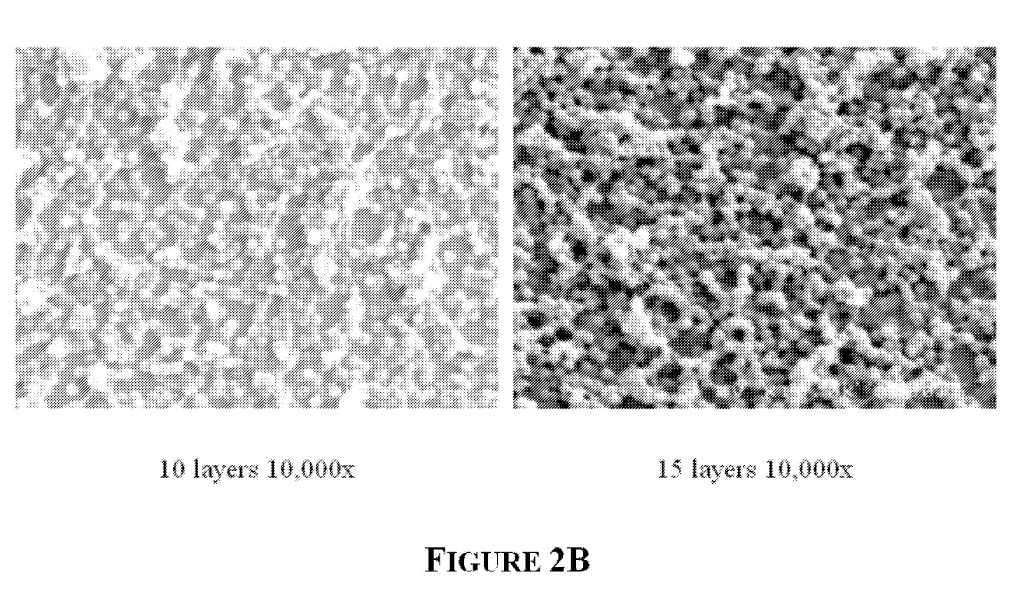

After calculating the area of the nanoparticles, it was concluded that the percentage of nanoparticles adhering to the surface of the steel is optimal after dipping a stainless steel sheet for at least five layers of polyelectrolytes. Each dipping took 5 minutes. Exemplary coatings are shown in FIG. 2 to illustrate surface coverage as a function of number of layers. FIG. 3 shows a graph, which illustrates the relationship of percentage of nanoparticles and number of layers at different magnifications.

### 2. Surface Coverage of Nanoparticles as a Function of Nanoparticle Concentration

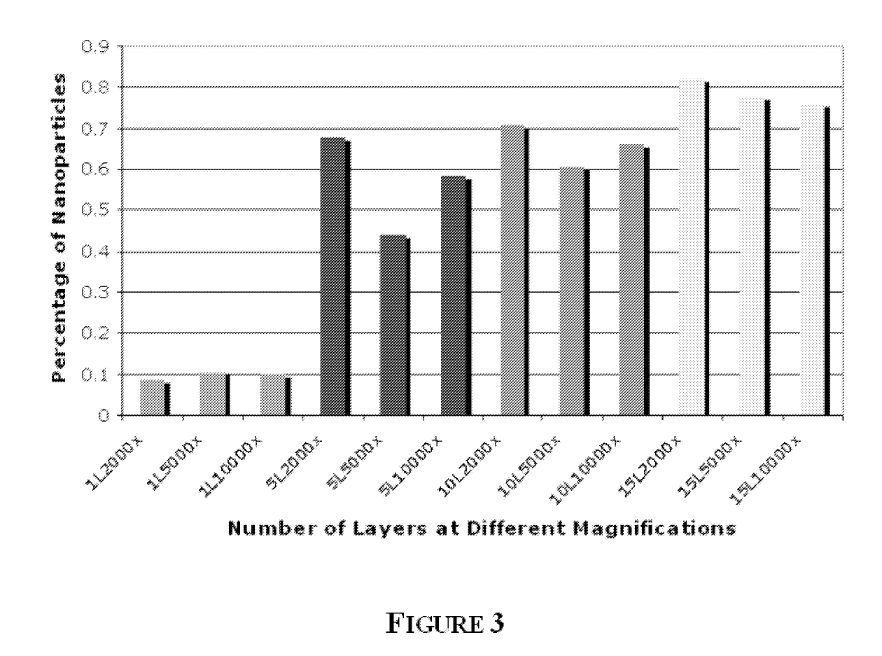

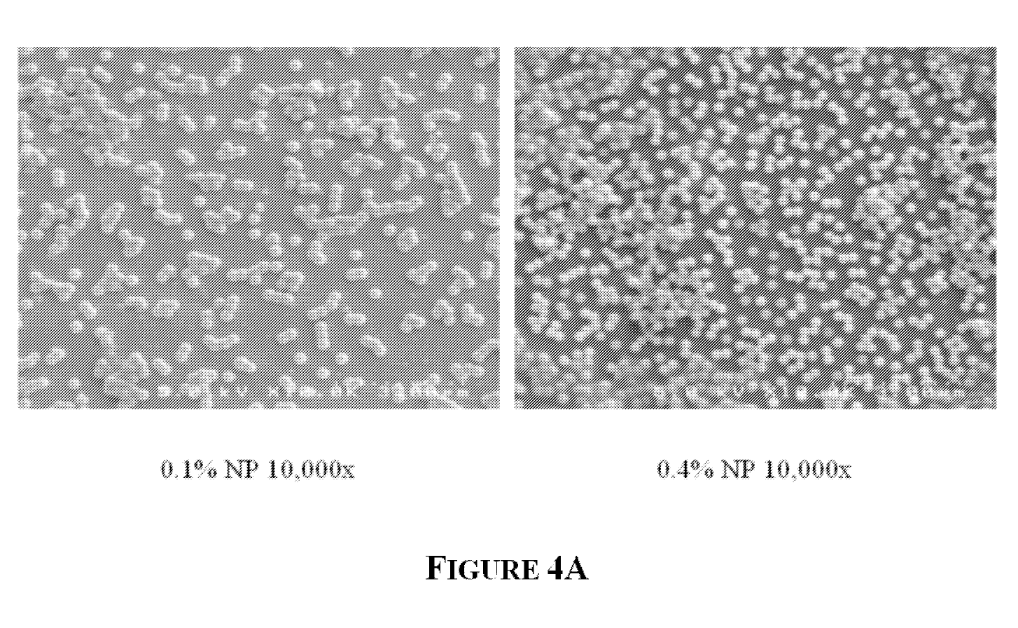

After analysis, the data indicated that dipping stainless steel sheets in 0.4% of NP concentration has the highest percentage of nanoparticles adhering to the sample. Stainless steel stents were dipped in the NP solution for ten minutes. Exemplary coatings are shown in FIG. 4 to illustrate percentage of nanoparticles as a function of nanoparticle concentration. FIG. 5 shows a graph, which illustrates the relationship of percentage of nanoparticles and nanoparticle concentration.

### 3. Surface Coverage of Nanoparticles as a Function of Dipping Time

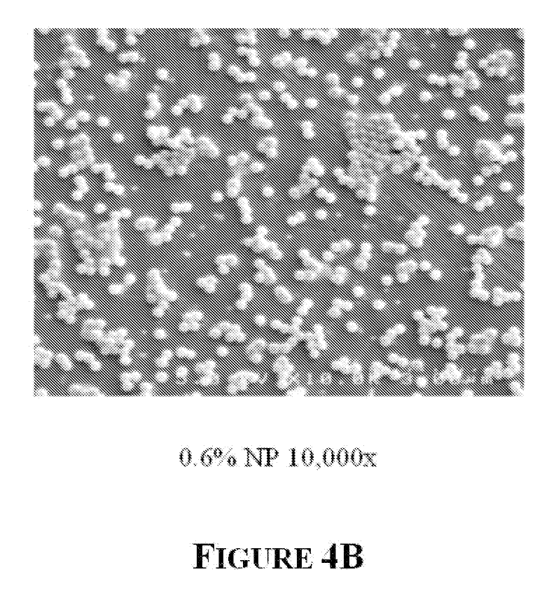

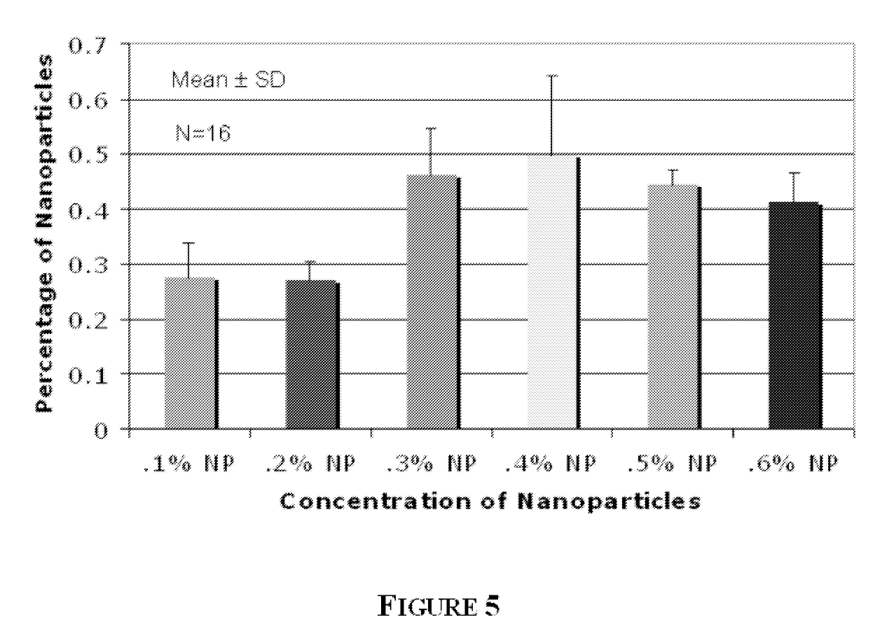

After analysis, the data indicated that dipping stainless steel samples into nanoparticles for 20 minutes obtained the optimal result with the highest amount of nanoparticles adhering to the surface. Samples were dipped in polycation (PC) solution for 10 minutes, and sonicated after the polyanion (PA) layer. A 0.1% NP concentration was used. FIG. 6 to illustrate percentage of nanoparticles as a function of dipping time. FIG. 7 shows a graph, which illustrates the relationship of percentage of nanoparticles and dipping time.

### 4. Summary

Dipping stainless steel stents in at least 5 layers of polyelectrolyte solutions produced the largest amount of nanoparticles adhering to the surface. The data indicates that a 0.4% NP solution with 5 or more layers at ten to twenty minutes of dipping time, in general, yields the greatest amount of nanoparticles adhering to the surface of the stainless steel sheets. 0.4% solution of nanoparticles provided the best coverage on the surface. Without wishing to be bound by theory, it is believed that the 0.4% concentration of NP solution represented the saturation point of NP onto the surface, which can explain why a higher concentration of NP did not necessarily cause more NP to adhere to the surface. Immersing the stainless steel sheets in PA solution for 10 to 20 minutes was optimal. After 20 minutes the NPs seem to dislodge themselves from the surface and return to the solution. Without wishing to be bound by theory, it is believed that one reason why there were not as many nanoparticles after one hour can be due to the restoration of entropy and disruption of the strong attraction forces between the layers.

### 5. REFERENCES

Pertinent literature references known to those of skill in the art that can be helpful in understanding the various aspects of the disclosed invention(s) include the following: G. Decher, Science 277, 1232 (Aug. 29, 1995); D. M. DeLongchamp, P. T. Hammond, Langmuir 20, 5403 (Jun. 22, 2004); P. T. Hammond, Macromolecules 28, 7569 (1995); P. T. Hammond, Adv. Mater. 16, 1271 (Aug. 4, 2004); B. Thierry et al., Adv. Mater. 17, 826 (Apr. 4, 2005); B. Thierry et al., J Am Chem Soc 127, 1626 (Feb. 16, 2005); B. Thierry et al., Biomacromolecules 4, 1564 (November-December, 2003); B. Thierry et al., J Am Chem Soc 125, 7494 (Jun. 25, 2003); and K. C. Wood et al., Langmuir 21, 1603 (Feb. 15, 2005).

### C. COATED SUBSTRATES

In one aspect, the invention relates to coated substrates. In one aspect, a layered polymeric coating can be prepared and can be suitable for use with implantable devices, for example a stent.

### 1. Mutilayer Coatings

In one aspect, the invention relates to a coated substrate comprising a substrate having a surface, a cationic polymer layer adjacent the surface of the substrate, wherein the cationic polymer layer comprises at least one residue of a first compound having the structure:

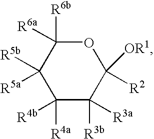

wherein R¹ is hydrogen or alkyl; wherein R², R^{3a}, R^{3b}, R^{4a}, R^{4b}, R^{5a}, R^{5b}, R^{6a}, and R^{6b} are, independently, hydrogen, hydroxyl, alkyl, aryl, alkoxy, carboxyl, ester, amino, or amide, with the provisos that at least one of R², R^{3a}, R^{3b}, R^{4a}, R^{4b}, R^{5a}, R^{5b}, R^{6a}, and R^{6b} is amino and that at least one of R², R^{3a}, R^{3b}, R^{4a}, R^{4b}, R^{5a}, R^{5b}, R^{6a}, and R^{6b} is hydroxyl or alkoxy; and an anionic polymer layer adjacent the cationic layer, wherein the anionic polymer layer comprises at least one residue of a compound having the structure:

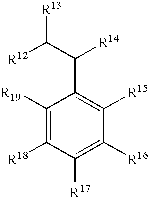

wherein R¹², R¹³, and R¹⁴ are, independently, hydrogen, alkyl, aryl, carboxyl, or ester; and wherein R¹⁵, R¹⁶, R¹⁷, R¹⁸, and R¹⁹ are, independently, hydrogen, alkyl, aryl, alkoxy, amino, amide, carboxyl, or ester, with the proviso that at least one of R¹⁵, R¹⁶, R¹⁷, R¹⁸, and R¹⁹ is SO₃R¹¹, wherein R¹¹ is hydrogen or alkyl. In a further aspect, the outermost polymer layer has a surface having fractal characteristics.

### a. Substrate

In one aspect, the substrate is an implant, which can be optionally substantially devoid of cells or tissue. In a further aspect, the substrate is a stent, an artificial joint, an artificial organ, a bone screw, a bone plate, or a tissue. In one aspect, the substrate comprises a material selected from stainless steel, cobalt-chromium alloy, titanium, Nitinol, ceramic, and polymer.

### b. Anionic Layer

In one aspect, the anionic polymer layer is positioned between the surface and the cationic polymer layer. In a further aspect, a second anionic polymer layer is positioned between the surface and the cationic polymer layer. In a further aspect, the anionic polymer layer comprises a polymer having the structure:

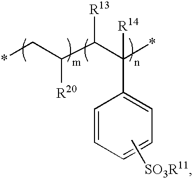

wherein R²⁰ is hydrogen, alkyl, or aryl; wherein m is zero or a positive integer; and wherein n is zero or a positive integer.

In a yet further aspect, the anionic polymer layer comprises one or more of polystyrene sulfonate, poly(acrylic acid), poly(methacrylic acid), substituted poly(phosphazene), poly(vinyl alcohol), heparin sulfate, chondroitin sulfate, dermatan sulfate, heparin, poly(aspartic acid), poly(tyrosine), copolymers of aspartic acid and tyrosine, other negatively charged poly amino acids, or dextrans. The anionic polymer layer, in a further aspect, comprises a self-assembled peptide layer.

In a still further aspect, the coated substrate can further comprise a second anionic polymer layer between the cationic polymer layer and the surface of the substrate. For example, the outer polymer layer can be an anionic polymer layer. In a still further aspect, the anionic polymer layer further comprises at least one of poly(styrene sulfonate), hyaluronic acid, alginate, or poly(glutamic acid).

### c. Cationic Layer

In one aspect, the cationic polymer layer is in contact with the surface of the substrate. The cationic polymer layer, in a further aspect, comprises a self-assembled peptide layer. In a further aspect, the cationic polymer layer comprises a polymer having the structure:

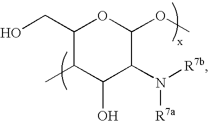

wherein R^{7a} and R^{7b} are independently hydrogen, alkyl, or acyl; wherein x is a positive integer. In a yet further aspect, the cationic polymer layer comprises a polymer having at least one residue of a compound comprising the structure:

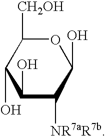

In a still further aspect, the cationic polymer layer comprises a polymer having the structure:

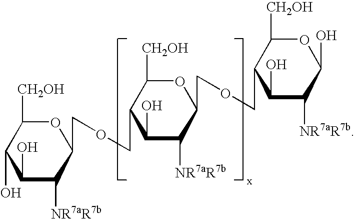

In a further aspect, the cationic polymer layer comprises poly-D-glucosamine.

In a further aspect, the cationic polymer layer comprises a polymer having at least one residue comprising the structure:

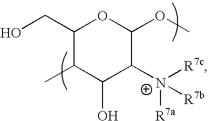

wherein R^{7a}, R^{7b}, and R^{7c} are, independently, hydrogen, alkyl, or acyl.

In a still further aspect, the coated substrate can further comprise a second cationic polymer layer adjacent the anionic layer. For example, the outer polymer layer can be a cationic polymer layer. In a further aspect, the cationic polymer layer further comprises at least one of chitosan, chitin, poly(L-lysine), poly(histidine), poly(imidazole), or poly(allylamines).

In one aspect, the coated substrate can further comprise at least one additional cationic polymer layer and at least one additional anionic polymer layer, thus having at least four total polymer layers.

### d. Payloads

The surface coatings can further comprise materials, referred to as payloads, which can impart functionality to the surface of the substrate. For example, in one aspect, the at least one polymer layer further comprises a payload comprising at least one imaging agent, at least one magnetically active agent, at least one pharmaceutically active agent, at least one biologically active agent, at least one functionalized polymeric nanoparticle, or at least one functionalized lipid nanoparticle. In a further aspect, the payload can be associated with the at least one polymer layer via ionic interactions, covalent interactions, coordination interactions, hydrophobic interactions, or electrostatic interactions. In a yet further aspect, the payload can be associated with the at least one polymer layer via sugar-inclusion complexation, supramolecular complexation, and/or streptavidin-biotin interactions.

In one aspect, the at least one polymer layer is a cationic polymer layer or an anionic polymer layer. In a further aspect, the payload can be associated with the at least one polymer layer via ionic interactions, covalent interactions, coordination interactions, electrostatic interactions, hydrophobic interactions, or hydrophilic interactions. In a yet further aspect, the payload can be associated with the at least one polymer layer via sugar-inclusion complexation. In a still further aspect, the payload can be associated with the at least one polymer layer via electrostatic interactions.

In various aspects, the at least one polymer layer can be a cationic polymer layer or an anionic polymer layer. In one aspect, the at least one polymer layer can be one or more imaging agents, one or more magnetically active agents, one or more biologically active agents, or one or more pharmaceutically active agents or a mixture thereof. In a further aspect, the payload comprises at least one imaging agent selected from Gadolinium-DTPA, a quantum dot, a gold nanoparticle, a silicon nanoparticle, iron oxide, magnetite (Fe₃O₄), Ferrodex, and Iron oxide coated with dextran.

In a yet further aspect, the payload comprises at least one magnetically active agent selected from Magnetite, Maghemite, Jacobsite, Trevorite, Magnesioferrite, Pyrrhotite, Greigite, Feroxyhyte, Iron, Nickel, Cobalt, Awaruite, Wairauite, Manganese salts, and mixtures thereof. In a further aspect, the payload comprises at least one pharmaceutically active agent selected from a steroid, a xanthine, a beta-2-agonist bronchodilator, an anti-inflammatory agent, an analgesic agent, a calcium antagonist, an angiotensin-converting enzyme inhibitors, a beta-blocker, a centrally active alpha-agonist, an alpha-1-antagonist, an anticholinergic/antispasmodic agent, a vasopressin analogue, an antiarrhythmic agent, an antiparkinsonian agent, an antiangina/antihypertensive agent, an anticoagulant agent, an antiplatelet agent, a sedative, an ansiolytic agent, a peptidic agent, a biopolymeric agent, an antineoplastic agent, a laxative, an antidiarrheal agent, an antimicrobial agent, an antifungal agent, a vaccine, a protein, a nucleic acid, a compound comprising a nucleic acid, the combination of a radiosensitizer and a chemotherapeutic, paclitaxel, anti-inflammatory, NSAID, and antibodies and antibody fragments. In a further aspect, the payload comprises at least one biologically active agent selected from an oligonucleotide, a plasmid DNA, a protein, and a peptide.

In one aspect, the at least one polymer layer further comprises a payload comprising at least one nanoparticle and/or at least one microparticle or a mixture thereof. That is, the payload can be at least one nanoparticle and/or at least one microparticle and/or the payload can include at least one nanoparticle and/or at least one microparticle. In a yet further aspect, the payload comprises at least one nanoparticle selected from a quantum dot, a gold nanoparticle, and a silicon nanoparticle, gold nanocapsule, silver colloids, a silica nanoparticle, functionalized silica nanoparticle, and titanium oxide nanoparticle.

### 2. Composite Coated Substrates

In one aspect, the invention relates to a coated substrate comprising a substrate having a surface, a cationic polymer layer adjacent the surface of the substrate, an anionic polymer layer adjacent the cationic polymer layer, and at least one nanoparticle and/or microparticle positioned within the anionic polymer layer. In a further aspect, the cationic polymer layer comprises at least one residue of a first compound having the structure:

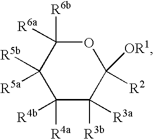

wherein R¹ is hydrogen or alkyl; wherein R², R^{3a}, R^{3b}, R^{4a}, R^{4b}, R^{5a}, R^{5b}, R^{6a}, and R^{6b} are, independently, hydrogen, hydroxyl, alkyl, aryl, alkoxy, carboxyl, ester, amino, or amide, with the provisos that at least one of R², R^{3a}, R^{3b}, R^{4a}, R^{4b}, R^{5a}, R^{5b}, R^{6a}, and R^{6b} is amino and that at least one of R², R^{3a}, R^{3b}, R^{4a}, R^{4b}, R^{5a}, R^{5b}, R^{6a}, and R^{6b} is hydroxyl or alkoxy; and wherein the anionic polymer layer comprises at least one residue of a compound having the structure:

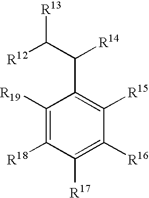

wherein R¹², R¹³, and R¹⁴ are, independently, hydrogen, alkyl, aryl, carboxyl, or ester; and wherein R¹⁵, R¹⁶, R¹⁷, R¹⁸, and R¹⁹ are, independently, hydrogen, alkyl, aryl, alkoxy, amino, amide, carboxyl, or ester, with the proviso that at least one of R¹⁵, R¹⁶, R¹⁷, R¹⁸, and R¹⁹ is SO₃R¹¹, wherein R¹¹ is hydrogen or alkyl. In a yet further aspect, the outermost polymer layer has a surface having fractal characteristics

In one aspect, the cationic polymer layer comprises at least one of chitosan, chitin, poly(L-lysine), or poly(allylamines). In a further aspect, the cationic polymer layer comprises a self-assembled peptide layer. In one aspect, the anionic polymer layer comprises at least one of poly(styrene sulfonate), hyaluronic acid, alginate, or poly(glutamic acid). In a further aspect, the anionic polymer layer comprises a self-assembled peptide layer. In one aspect, the cationic polymer layer comprises chitosan and the anionic polymer layer comprises poly(styrene sulfonate). In a still further aspect, one or both of the cationic polymer layer and the anionic polymer layer further comprises a payload comprising at least one imaging agent, at least one magnetically active agent, at least one pharmaceutically active agent, at least one biologically active agent, at least one functionalized polymeric nanoparticle, and/or at least one functionalized lipid nanoparticle. In a further aspect, the at least one nanoparticle or microparticle comprises at least one nanoparticle selected from a quantum dot, a gold nanoparticle, and a silicon nanoparticle. In a further aspect, the at least one nanoparticle or microparticle comprises at least one microparticle selected from a gold microparticle, or a silicon microparticle.

### D. METHODS FOR MAKING COATED SUBSTRATES

In one aspect, the invention relates to methods that can be used to provide the disclosed coated surfaces. Accordingly, the surface coatings, layers, substrates, payloads, particles, compounds, residues, and moieties disclosed herein can be used in connection with the disclosed methods.

In a further aspect, the invention relates to a method of making a coated substrate comprising the steps of providing a substrate having a surface; contacting the surface with an ionic polymer solution, thereby disposing an ionic polymer layer adjacent to the surface; and contacting the ionic polymer layer with a counterionic polymer solution, thereby disposing a counterionic polymer layer adjacent to the ionic polymer layer, wherein one of the ionic polymer layer and the counterionic polymer layer comprises at least one residue of a first compound having the structure:

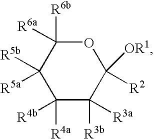

wherein R¹ is hydrogen or alkyl; wherein R², R^{3a}, R^{3b}, R^{4a}, R^{4b}, R^{5a}, R^{5b}, R^{6a}, and R^{6b} are, independently, hydrogen, hydroxyl, alkyl, alkoxy, carboxyl, ester, amino, or amide, with the provisos that at least one of R², R^{3a}, R^{3b}, R^{4a}, R^{4b}, R^{5a}, R^{5b}, R^{6a}, and R^{6b} is amino and that at least one of R², R^{3a}, R^{3b}, R^{4a}, R^{4b}, R^{5a}, R^{5b}, R^{6a}, and R^{6b} is hydroxyl or alkoxy; and wherein the other of the ionic polymer layer and the counterionic polymer layer comprises at least one residue of a compound having the structure:

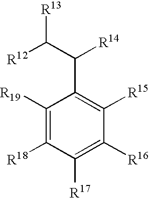

wherein R¹², R¹³, and R¹⁴ are, independently, hydrogen, alkyl, carboxyl, or ester; and wherein R¹⁵, R¹⁶, R¹⁷, R¹⁸, and R¹⁹ are, independently, hydrogen, alkyl, alkoxy, amino, amide, carboxyl, or ester, with the proviso that at least one of R¹⁵, R¹⁶, R¹⁷, R¹⁸, and R¹⁹ is SO₃R¹¹, wherein R¹¹ is hydrogen or alkyl.

In one aspect, the outermost polymer layer has a surface having fractal characteristics. In a further aspect, the ionic layer is anionic and the counterionic layer is cationic. In a yet further aspect, the cationic polymer layer comprises poly-D-glucosamine.

In a further aspect, the anionic polymer layer comprises one or more of polystyrene sulfonate, poly(acrylic acid), poly(methacrylic acid), substituted poly(phosphazene), poly(vinyl alcohol), heparin sulfate, chondroitin sulfate, dermatan sulfate, heparin, pol(aspartic acid), poly(tyrosine), copolymers of aspartic acid and tyrosine, other negatively charged poly amino acids, dextrans, or aliginic acid.

In a further aspect, the anionic polymer layer comprises a polymer having the structure:

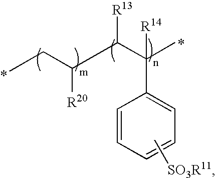

wherein R²⁰ is hydrogen, alkyl, or aryl; wherein m is zero or a positive integer; and wherein n is zero or a positive integer. In a yet further aspect, the anionic polymer layer comprises polystyrene sulfonate.

In one aspect, the cationic polymer layer comprises a polymer having the structure:

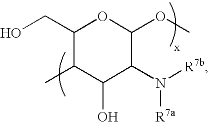

wherein R^{7a} and R^{7b} are independently hydrogen, alkyl, or acyl; wherein x is a positive integer. In a yet further aspect, the cationic polymer layer comprises a polymer having at least one residue of a compound comprising the structure:

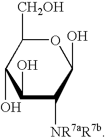

In a still further aspect, the cationic polymer layer comprises a polymer having the structure:

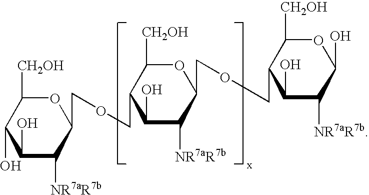

In a further aspect, the cationic polymer layer comprises poly-D-glucosamine. In a yet further aspect, the cationic polymer layer comprises a polymer having at least one residue comprising the structure:

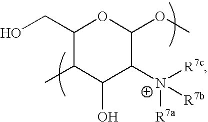

wherein R^{7a}, R^{7b}, and R^{7c} are, independently, hydrogen, alkyl, or acyl. A method of making a coated substrate comprising the steps of providing a substrate having a surface; contacting the surface with an ionic polymer solution, thereby disposing an ionic polymer layer adjacent to the surface; and contacting the ionic polymer layer with a counterionic polymer solution, thereby disposing a counterionic polymer layer adjacent to the ionic polymer layer, wherein one or both of the ionic polymer solution and the counterionic polymer solution further comprises least one nanoparticle or microparticle.

In one aspect, the ionic polymer solution is a cationic polymer solution and the counterionic polymer solution is an anionic polymer solution. In a further aspect, the cationic polymer solution comprises chitosan. In a yet further aspect, the anionic polymer solution comprises poly(styrene sulfonate).

In a further aspect, one or both of the ionic polymer solution and the counterionic polymer solution further comprises a payload comprising at least one imaging agent, at least one magnetically active agent, at least one pharmaceutically active agent, at least one biologically active agent, at least one functionalized polymeric nanoparticle, or at least one functionalized lipid nanoparticle. In one aspect, the anionic polymer solution further comprises at least one nanoparticle or microparticle. In a yet further aspect, the at least one nanoparticle or microparticle comprises at least one nanoparticle selected from a quantum dot, a gold nanoparticle, and a silicon nanoparticle.

### E. SURFACE FUNCTIONALIZED NANOPARTICLES

In one aspect, the nanoparticles of the present invention comprise surface functionalized nanoparticles prepared from degradable polymers. Such particles can be used in combination with any of the other nanoparticles, coating techniques, and methods described herein and the present invention is not intended to be limited to any particular particle, coating, method, or combination thereof. In one aspect, a nanoparticle having a desired surface functionality can be prepared by, for example, entrapment of one or more polyelectrolytes using a rapid phase inversion and solidification technique. In a specific aspect, an aqueous phase can be combined with a ternary system composed of, for example, a polymer and two solvents having a combined polarity close to water, so as to create an instantaneous stable microemulsion. Such a thermodynamically stable microemulsion can serve as a matrix for the nucleation and growth of polymer nanoparticles. If one or more of the phases, for example, the aqueous phase, in which subsequent nucleation and growth of polymeric nanoparticles occurs, comprises a desirable soluble species, the desirable species can be entrapped in the solidifying polymeric particulate phase, resulting in a functionalized nanoparticle. Such a single step technique can, for example, reduce and/or eliminate the need for surfactants and other stabilization aids required in conventional nanoparticle preparation processes.

The polymer for a surface functionalized nanoparticle can comprise any polymeric material suitable for use in the rapid phase inversion and solidification technique described herein or in variants thereof. In one aspect, the polymeric material comprises any biodegradable polymer than can decompose under normal physiological conditions to produce materials having little or no toxicity. In another aspect, the polymer comprises a copolymer of, for example, lactic acid and glycolic acid. In a specific aspect, the polymer comprises a poly(lactic-co-glycolic acid) (PLGA), such as, for example, RESOMER® RG502 and/or RG503 PLGA, available from Boehringer Ingelheim Pharma GmbH & Co, Germany. The composition and molar ratios of any particular monomeric, oligomeric, and/or polymeric material comprising a portion of, for example, a copolymer, can vary depending upon the desired properties of the resulting nanoparticle. In a specific aspect, the molar ratio of lactide to glycolide in a PLGA polymer can range from about 48:52 to about 52:48, for example 48:52; 49:51; 50:50; 51:49; or 52:48. In other aspects, the molar ratio of lactide to glycolide can be less than or greater than the values described herein. In various aspects, a polymer material can comprise a PLGA, poly(styrene sulfonate), poly(acrylic acid), poly(lactic acid), poly(lysine hydrochloride), poly(ethylene glycol), heparin, poly(ethylene oxide), or a combination thereof. Any one or more polymer materials can optionally be purified by, for example, precipitation from a suitable solvent system prior to use. The specific molecular weight of a particular polymer material can vary depending upon, for example, the desired size and density of a resulting nanoparticle. In one aspect, a polymer has a molecular weight of about 30,000. In another aspect, a polymer has a molecular weight of about 70,000.

The solvent pairs used in the rapid inversion and solidification techniques described herein can be any suitable solvent pair that have a combined polarity similar to or substantially similar to water. In one aspect, a solvent pair comprises tetrahydrofuran (THF) and acetone. In another aspect, a solvent pair comprises 1-methyl-2-pyrolidone (NMP) and acetone. Other solvent pairs and/or combinations of solvents can also be used provided that such use can provide the instantaneous stable microemulsion described herein. The volumetric ratio of any one or more solvents in a solvent system can vary and can be optionally optimized to yield nanoparticles of varying sizes. Nanoparticle produced from the techniques described herein can range from about 70 to about 500 nm in size. In one aspect, the produced nanoparticles have an average size of about 250 nm. In other aspects, the size of nanoparticles can be less than about 70 or greater than about 500 nm. In yet another aspect, nanoparticles can be produced in various target sizes or ranges thereof, such as for example, about 250 nm, without the need for additional steric stabilization agents.

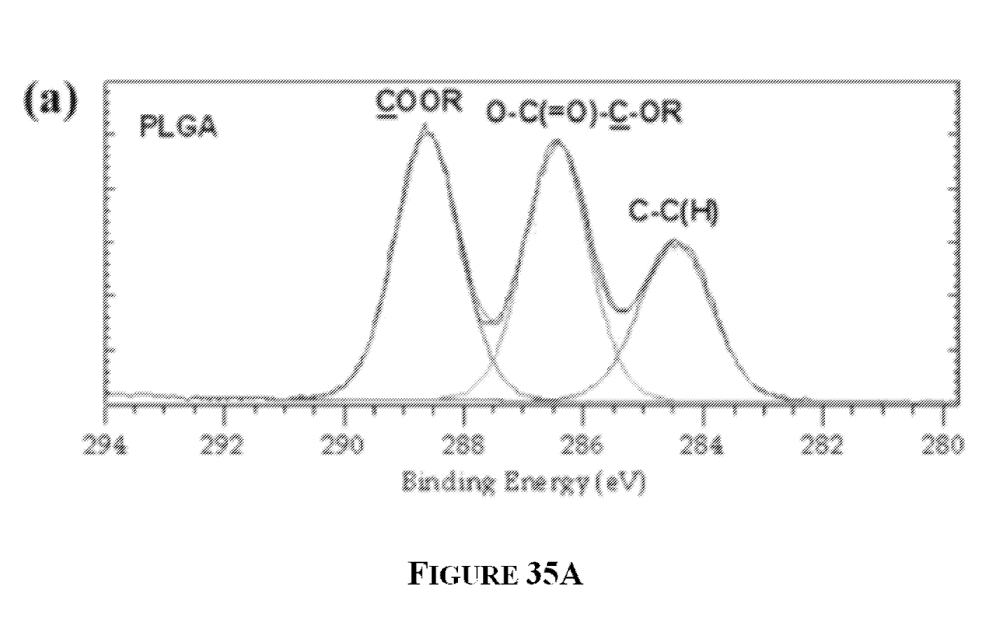

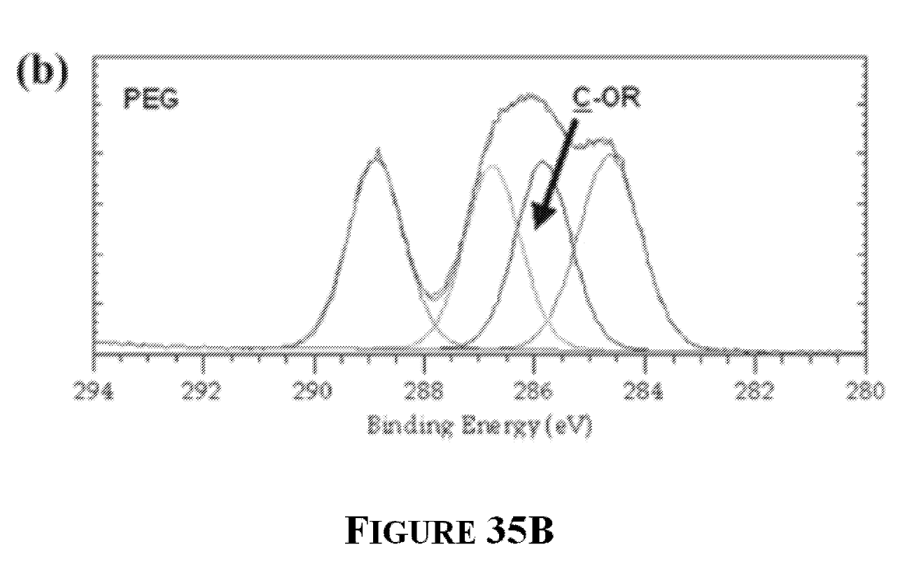

The concentration of a polymer in a solvent system (i.e., solvent pair) can be any suitable concentration that can provide a nanoparticle having properties suitable for the intended application. In one aspect, the polymer concentration ranges from about 2 to about 50 mg/mL. In another aspect, the polymer concentration rangers from about 10 to about 20 mg/mL. Functionalization of a nanoparticle can be performed by, for example, adding one or more polyelectrolytes or water soluble polymers, such as a poly(ethylene glycol) to the aqueous phase. The concentration of a polyelectrolyte or water soluble polymer, if used, can vary, depending upon the desired concentration of functional groups on a nanoparticle surface. In one aspect, the concentration of such a polyelectrolyte or water soluble polymer can be less than about 0.5 w/v %, or about 0.05 w/v %. The presence of an imparted functional group on a nanoparticle surface can be detected by, for example, measuring the zeta potential as a function of pH, by XPS analysis, other suitable surface analysis techniques, or combinations thereof. FIG. 34 illustrates the changes in zeta potential of an exemplary functionalized nanoparticle as a function of pH for various systems, including an unmodified PLGA nanoparticle (PLGA (P), a poly(styrene sulfonate) modified nanoparticle (P-PSS); a poly(acrylic acid) modified nanoparticle (P-PAA); a heparin modified nanoparticle (P-Hep); and a poly(lysine) modified nanoparticle (P-Lys). FIG. 34(b) illustrates scanning electron micrographs of PLGA nanoparticles modified with (A) poly(acrylic acid) and (B) poly(styrene sulfonate). The scale bar in each of the micrographs represents 100 nm. Further, FIG. 35 illustrates X-ray photoelectron spectroscopy (XPS) analysis data of: (a) unmodified PLGA nanoparticles, and (b) poly(ethylene glycol) modified nanoparticles. The arrow in FIG. 35(b) indicates the new peak due to the —O—CH₂ carbons from the poly(ethylene glycol).

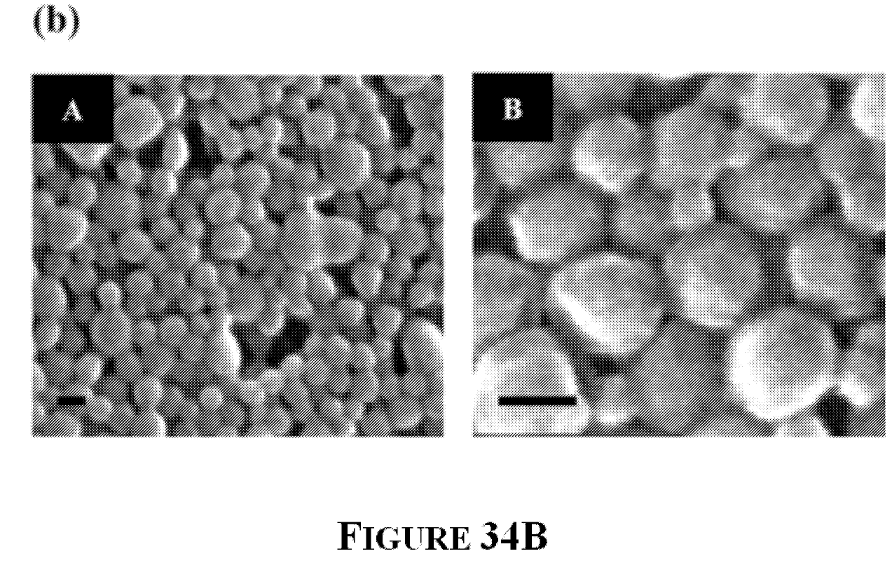

The nanoparticle suspension produced by the rapid phase inversion and solidification technique described herein can optionally be purified by one or more dialysis steps to, for example, remove organic components and any remaining, un-trapped, water soluble species, and/or concentrate the nanoparticle suspension. The size of nanoparticles produced using the rapid phase inversion technique can be controlled by, for example, adjusting the polarity of the solvent system. While not wishing to be bound by theory, it is believed that increased miscibility (e.g., with the aqueous phase) due to increased polarity of the solvent system should promote more rapid polymer-phase gelation. FIG. 33(a) illustrates the relationship between acetone volume fraction and average nanoparticle size. The viscosity of the aqueous phase can also affect the size of produced nanoparticles. For example, addition of glycerol to the aqueous phase, resulting in an increase in aqueous phase viscosity, as illustrated in FIG. 33(b), can result in a corresponding increase in nanoparticle size. Again, while not wishing to be bound by theory, increased aqueous phase viscosity is believed to impair water diffusion into the organic phase, making the sol-gel transition in the polymer phase less sharp.

In addition to or in alternative to a surface functional group, a nanoparticle, such as a PLGA nanoparticle, can comprise one or more payloads, such as a pharmaceutically active species. Such as payload can be contacted with and/or optionally attached to a nanoparticle after formation of the nanoparticle or can be incorporated simultaneously with formation of the nanoparticle, such as described for the polyelectrolytes and water soluble polymers described herein. In one aspect, a nanoparticle comprises a drug molecule. In other various aspects, a nanoparticle comprises one or more model drug molecules, such as, for example, fluorescein, fluorescein diacetate dye, bovine serum albumin, fluorescein isothiocyanate (FITC) tagged bovine serum albumin, horseradish peroxidase, or a combination thereof.

In a specific aspect, a nanoparticle can be surface functionalized with a poly(styrene sulfonate) by, for example, suspension in a poly(styrene sulfonate) solution. In a specific aspect, the poly(styrene sulfonate) solution can be about 0.3 w/v %. Such functionalized nanoparticles can be assembled on, for example, a stainless steel foil through a LBL assembly of poly(styrene sulfonate)-chitosan. In other aspects, a substrate surface can be coated with a plurality of nanoparticles comprising the same and/or different compositions, sizes, functionalities, and/or payloads.

By tailoring the nanoparticle surface and optional payload, one or more individual drugs can be delivered, wherein each drug can have a distinct release profile. In one aspect, a first nanoparticle can comprise a fluorescein molecule and a second nanoparticle can comprise a bovine serum albumin, horseradish peroxidase/bovine serum albumin, and horseradish peroxidase/fluorescein. In one aspect, a release profile can be tailored to deliver a drug for at least 5 days, for a plurality of weeks, or for a plurality of months. In another aspect, each of the one or more drugs and/or model drug molecules can have a distinct release profile over a period of from about 5 days to a plurality of months.

In one aspect, the present invention provides a coated substrate comprising at least one nanoparticle formed from a biodegradable polymer. In various aspects, the biodegradable polymer comprises at least one of a poly(lactic-co-glycolic acid), poly(styrene sulfonate), poly(acrylic acid), poly(lactic acid), poly(lysine hydrochloride), poly(ethylene glycol), heparin, poly(ethylene oxide), or a combination thereof. In a specific aspect, the biodegradable polymer comprises a poly(lactic-co-glycolic acid). In another aspect, the nanoparticle comprises at least one payload. In a specific aspect thereof, a payload comprises an imaging agent, a magnetically active agent, a pharmaceutically active agent, a biologically active agent, a functionalized polymer, a functionalized lipid, or a combination thereof. In another specific aspect, the nanoparticle comprises a bovine serum albumin, a horseradish peroxidase, a fluorescein, or a combination thereof.

In another aspect, the present invention provides a coated substrate comprising a substrate having a surface, a cationic polymer layer adjacent to the surface of the substrate, an anionic polymer layer adjacent to the cationic polymer layer, and at least one nanoparticle, wherein the at least one nanoparticle is comprises a biodegradable polymer. In a specific aspect, the nanoparticle comprises a poly(lactic-co-glycolic acid).

In yet another aspect, the present invention provides a method of making a nanoparticle comprising a biodegradable polymer, the method comprising contacting an aqueous phase with a ternary system, wherein the ternary system comprises a polymer and a solvent pair, so as to create a stable nanoparticle suspension. In a specific aspect, the polymer comprises at least one of a poly(lactic-co-glycolic acid), poly(styrene sulfonate), poly(acrylic acid), poly(lactic acid), poly(lysine hydrochloride), poly(ethylene glycol), heparin, poly(ethylene oxide), or a combination thereof. In another specific aspect, the solvent pair comprises at least one of THF/acetone, NMP/acetone, or a combination thereof.

In yet other aspects, the present invention provides a method for preparing a functionalized nanoparticle, the method comprising adding one or more species to the aqueous phase of the method described above, such that, during contacting, the species in the aqueous phase is at least partially incorporated into the matrix of the solidifying polymer. In a specific aspect, the species at least partially water soluble. In another specific aspect, the species is water soluble. In yet another specific aspect, the species comprises a polyelectrolyte, a water soluble polymer, or a combination thereof. In yet another specific aspect, the species comprises a payload, such as a pharmaceutically and/or biologically active molecule.

In yet other aspects, any of the compositions and/or methods described herein can comprise a nanoparticle comprising a biodegradable polymer, such as, for example, a PLGA.

### F. IMPLANTATION

In one aspect, the invention relates to a method of treating comprising the step of implanting a disclosed coated substrate or a product produced by a disclosed method into a subject, for example a patient. In a further aspect, the at least one polymer layer further comprises a payload comprising at least one imaging agent, the method further comprising the step of imaging the coated substrate.

### G. RADIO FREQUENCY ABLATION

In one aspect, the invention relates to a method of performing radio frequency ablation comprising the steps of providing the coated substrate of the disclosed coated substrate or the disclosed product, wherein the coated substrate or the product further comprises at least one metal nanoparticle or metal microparticle; and exposing the coated substrate or the product to radio frequency radiation. In a further aspect, the metal nanoparticle or the metal microparticle comprises gold. In a yet further aspect, the coated substrate of the coated substrate or the product produced is implanted within a subject.

In a further aspect, the disclosed radio frequency ablation methods can be used to remove tissue proximate to the substrate. The disclosed radio frequency ablation methods can also be used, for example, in combination with imaging methods.

### H. EXPERIMENTAL

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. GENERAL METHODS

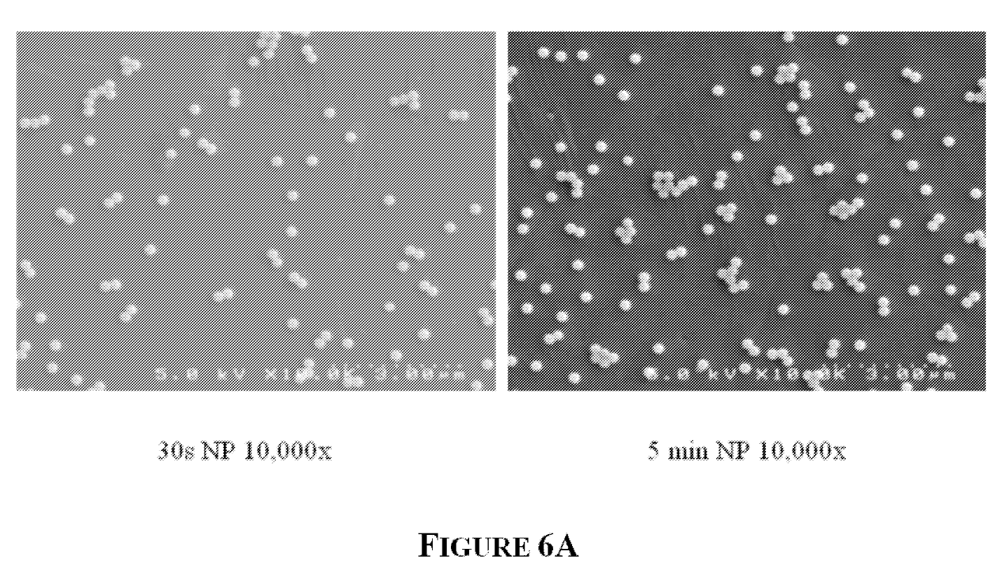

316L stainless steel samples, (the most common material used to manufacture stents) were dipped into the polyelectrolyte solutions, sonicated (high frequency sound waves used to dislodge any non-bonded NPs), and viewed with a microscope. Scion Image analysis software was used to calculate the percentage of nanoparticles incorporated onto the surface of the stainless steel samples. Samples were dipped into polyelectrolyte solutions, sonicated, and then viewed. A schematic illustrating a general procedure is shown in FIG. 8.

### 2. EXAMPLES

In the following examples, unless otherwise noted, all chitosan (CH) solutions are in 25% acetic acid with 0.14M NaCl deionized (DI) H₂O, and all poly(styrene sulfonate) (PSS) solutions are in 0.14M NaCl DI H₂O Unless otherwise described the nanoparticles used in the general experimentals were negatively charged PS latex nanoparticles. It is understood, however, that other micro- and nanoparticles of differing charge characteristics can be used, for example gold and gadolinium particles. It is also understood that potential impurities can be further minimized or eliminated by using glove boxes.

Typically, a stent was immersed in the listed solution prepared at the listed concentration for the listed time; the associated micrograph shows the resultant coating.

### a. EXAMPLE 1

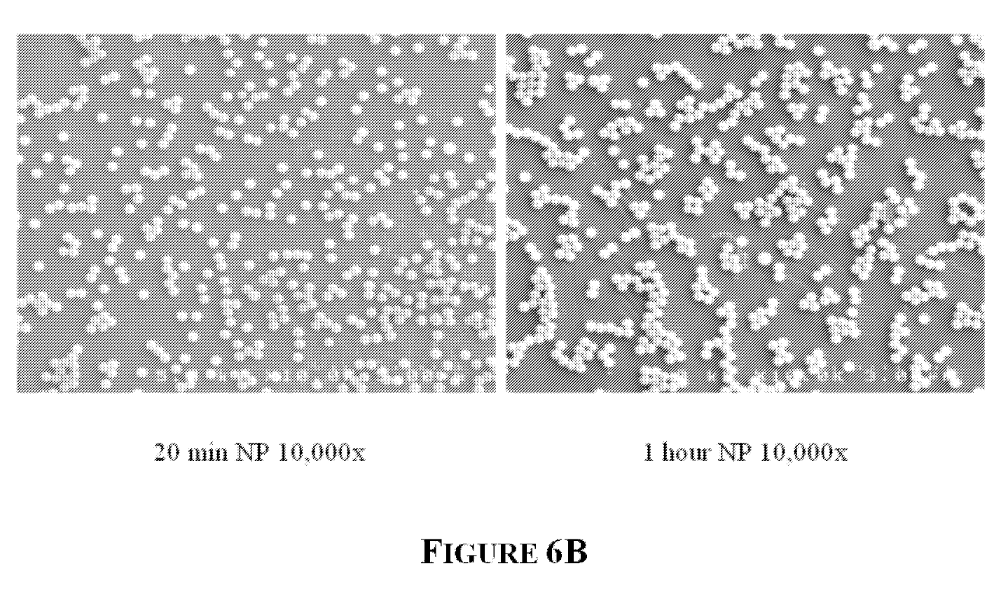

The first layer was CH (2 mg/ml; 10 min). The second layer PSS (2 mg/ml) w/nanoparticles with 0.5% nanoparticles for 5 min and sonicated afterwards at 21% intensity. The resultant micrograph is shown in FIG. 9.

### b. EXAMPLE 2

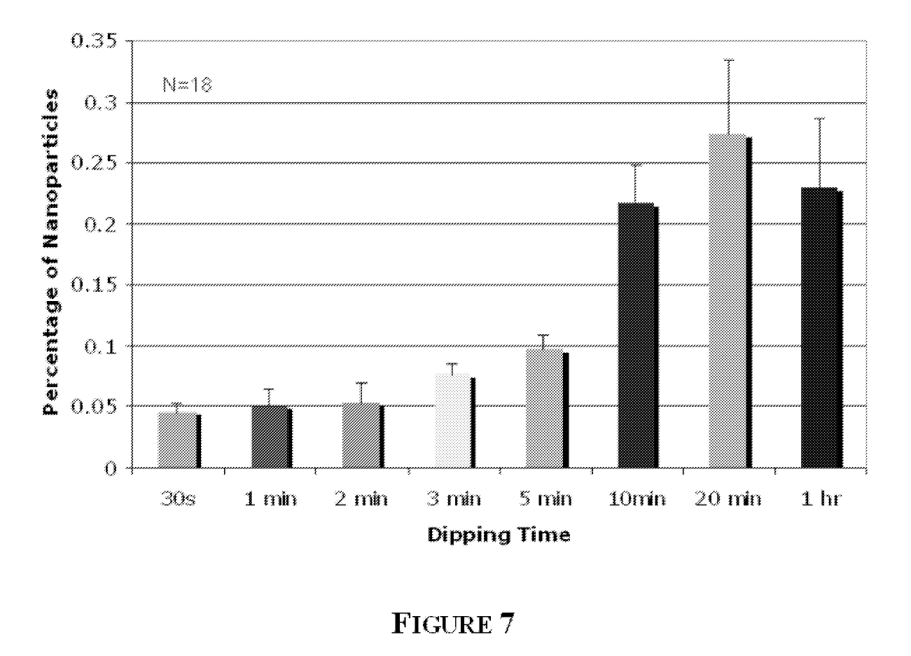

The first layer was CH (2 mg/ml; 10 min). The second layer PSS (2 mg/ml) w/nanoparticles with 0.5% nanoparticles for 5 min and sonicated afterwards at 21% intensity. The third layer was CH (5 min). The fourth layer was PSS (5 min) and sonicated afterwards. A total of 5 PSS layers were deposited. The resultant micrograph is shown in FIG. 10.

### C. EXAMPLE 3

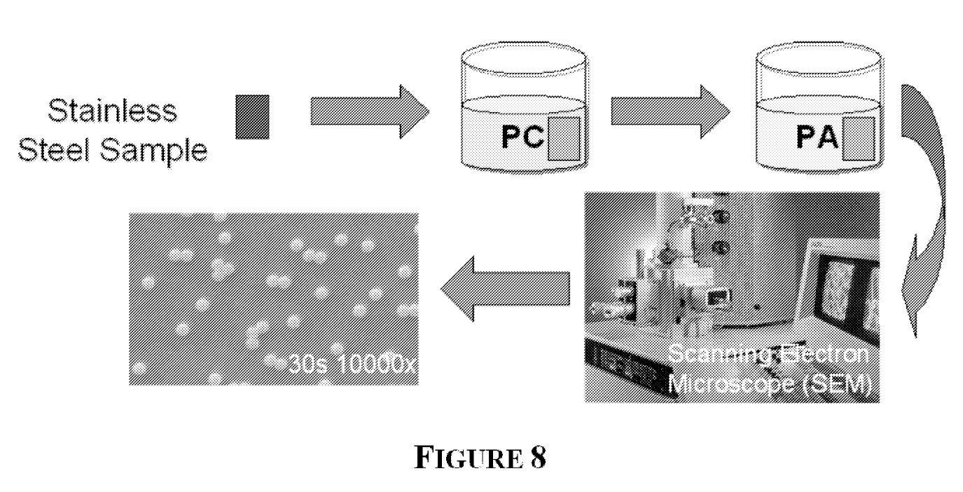

The first Layer was CH (2 mg/ml; 10 min). The second layer was PSS (2 mg/ml) w/nanoparticles with 0.5% nanoparticles for 5 min and sonicated afterwards at 21% intensity. The third layer was CH (5 min). The fourth layer was PSS layer (5 min)—sonicated afterwards. A total of 10 PSS layers were deposited. The resultant micrograph is shown in FIG. 11.

### d. EXAMPLE 4

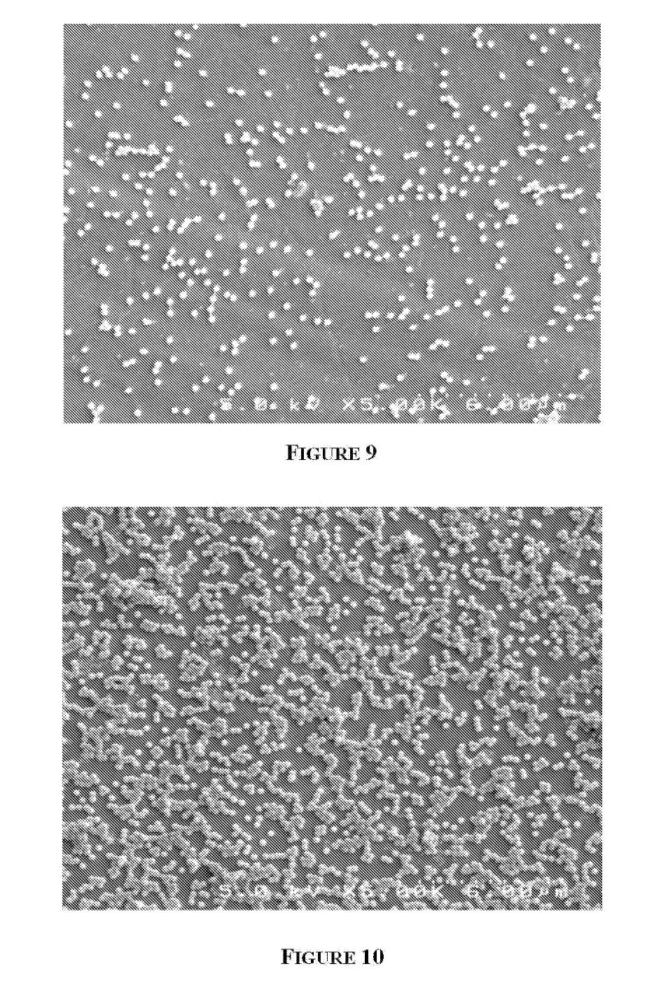

The first layer was CH (2 mg/ml; 10 min). The second layer was PSS (2 mg/ml) w/nanoparticles with 0.5% nanoparticles for 5 min sonicated afterwards at 21% intensity. The third layer was CH (5 min). The fourth layer was PSS (5 min)—sonicated afterwards. A total of 15 PSS layers were deposited. The resultant micrograph is shown in FIG. 12.

### e. EXAMPLE 5

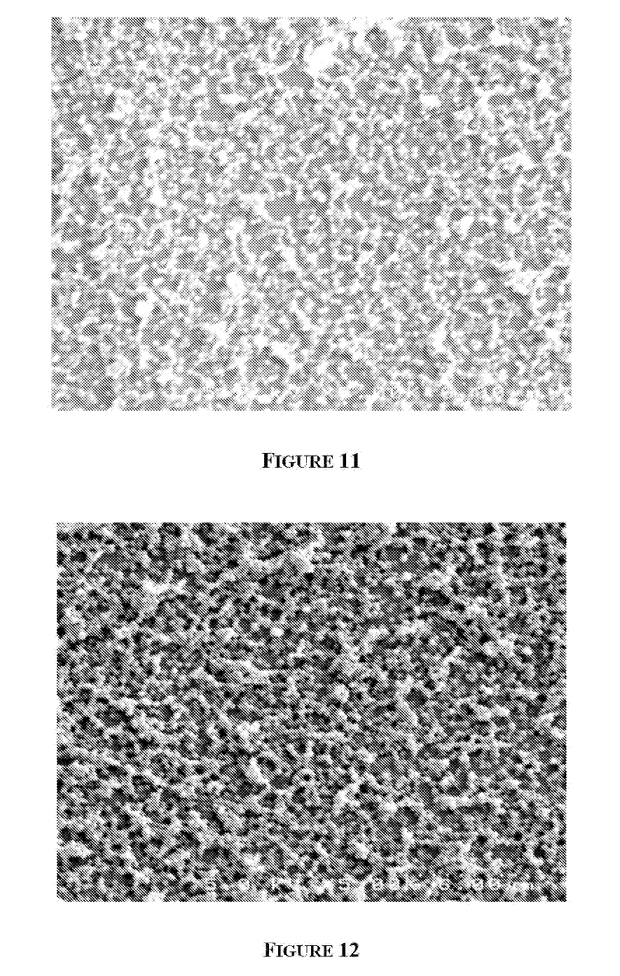

The first layer was CH (2 mg/ml; 10 min). The second layer was PSS (2 mg/ml) with 0.1% nanoparticle for 10 min. The resultant micrograph is shown in FIG. 13.

### f. EXAMPLE 6

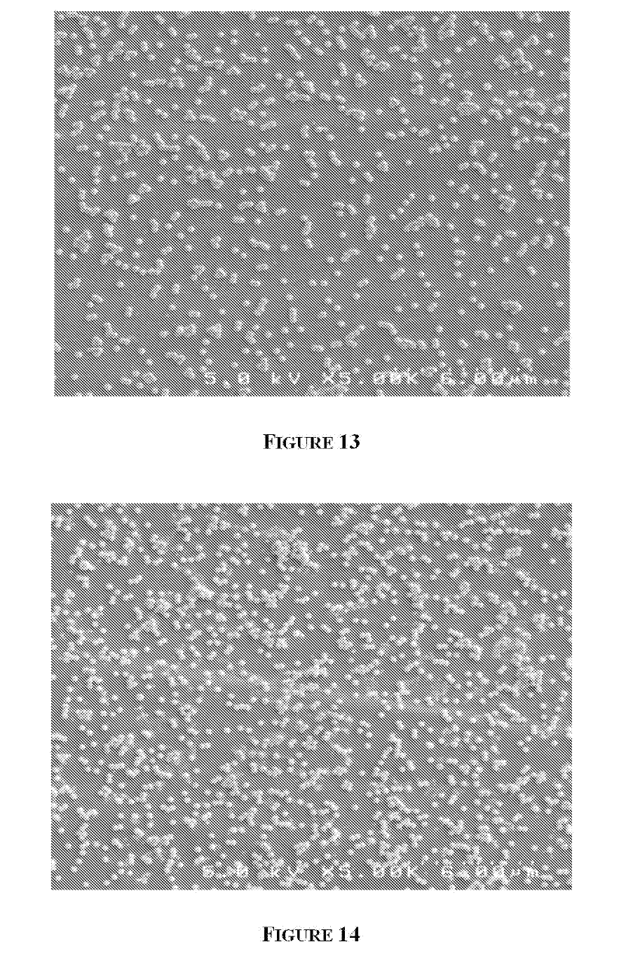

The first layer was CH (2 mg/ml; 10 min). The second layer was PSS (2 mg/ml) with 0.2% nanoparticle for 10 min. The resultant micrograph is shown in FIG. 14.

### g. EXAMPLE 7

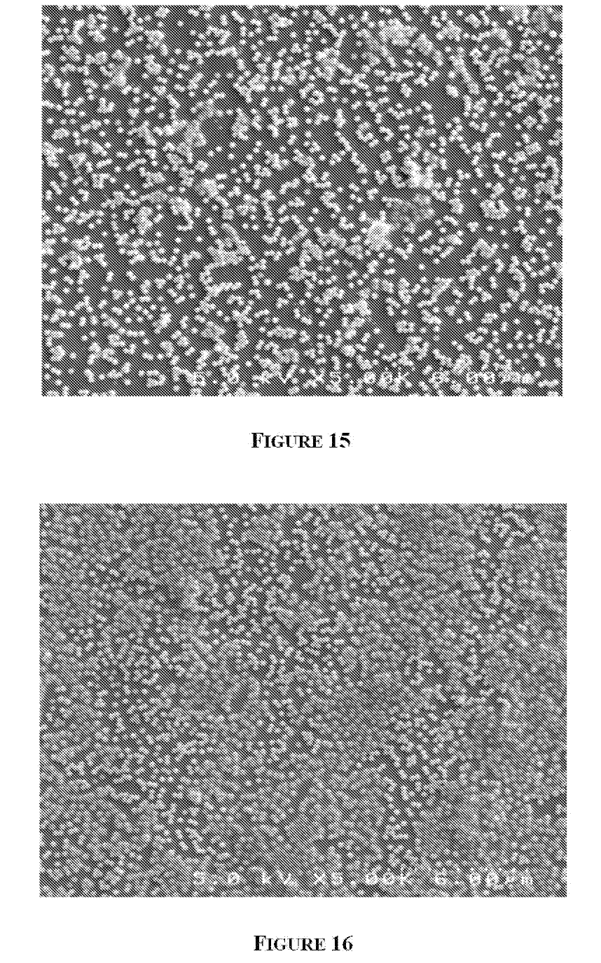

The first layer was CH (2 mg/ml; 10 min). The second layer PSS (2 mg/ml) with 0.3% nanoparticle for 10 min. The resultant micrograph is shown in FIG. 15.

### h. EXAMPLE 8

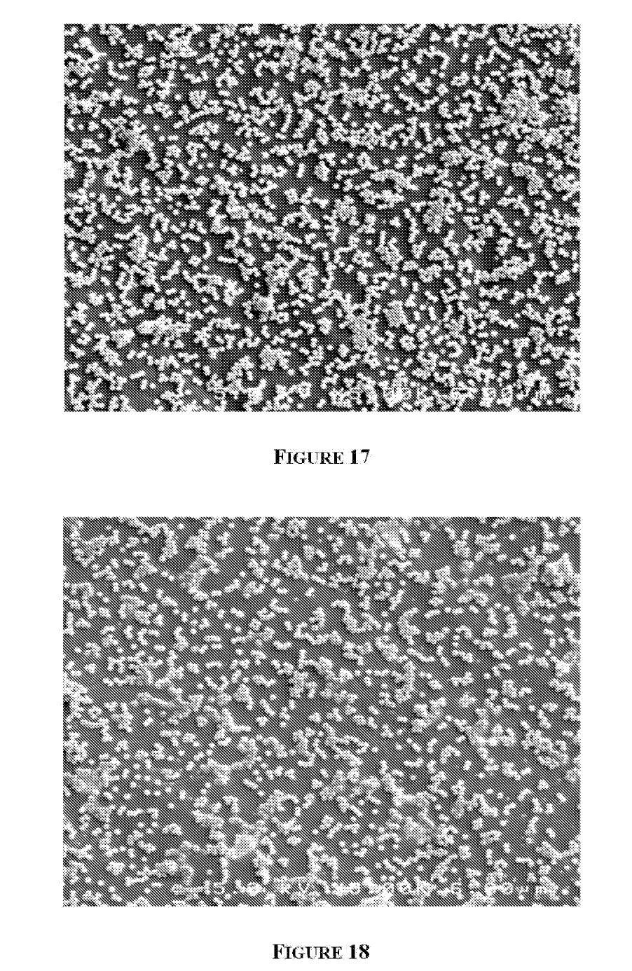

The first layer was CH (2 mg/ml; 10 min). The second layer was PSS (2 mg/ml) with 0.4% nanoparticle for 10 min. The resultant micrograph is shown in FIG. 16.

### i. EXAMPLE 9

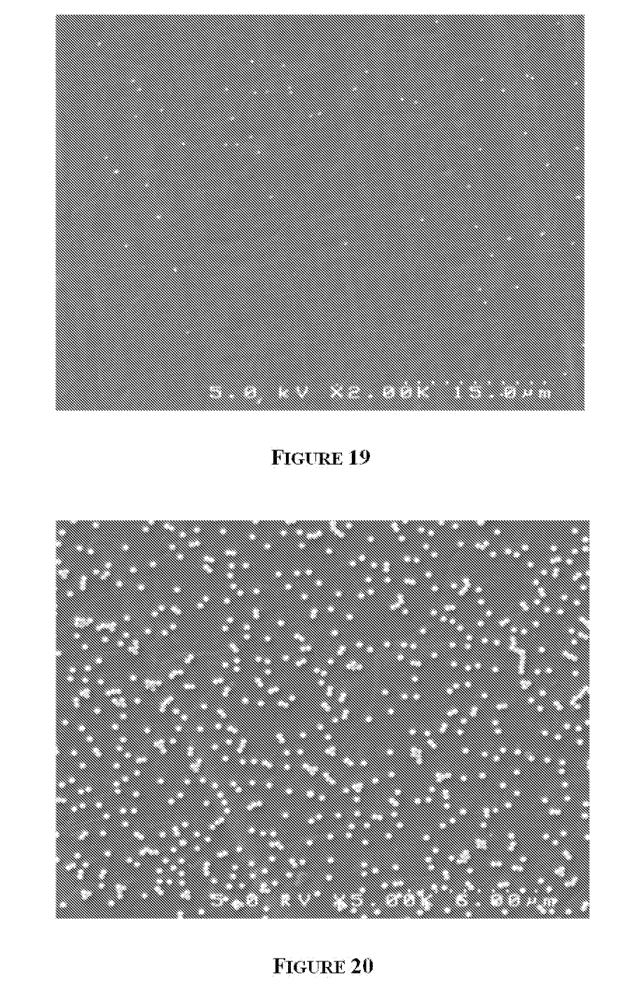

The first layer was CH (2 mg/ml; 10 min). The second layer was PSS (2 mg/ml) with 0.5% nanoparticle for 10 min. The resultant micrograph is shown in FIG. 17.

### j. EXAMPLE 10

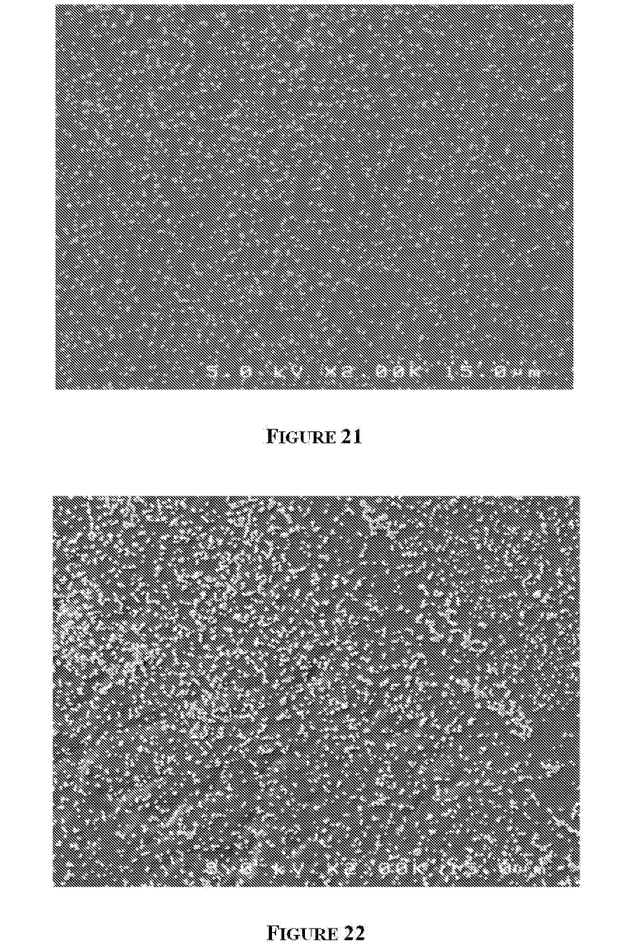

The first layer was CH (2 mg/ml; 10 min). The second layer was PSS (2 mg/ml) with 0.6% nanoparticle for 10 min. The resultant micrograph is shown in FIG. 18.

### k. EXAMPLE 11

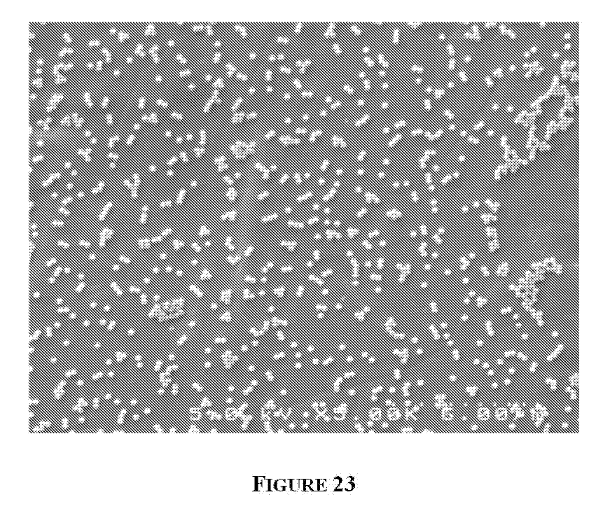

The first layer was CH (2 mg/ml; 10 min). The second layer was PSS (2 mg/ml) with 0.1% nanoparticle for 1 min. The resultant micrograph is shown in FIG. 19.

### l. EXAMPLE 12

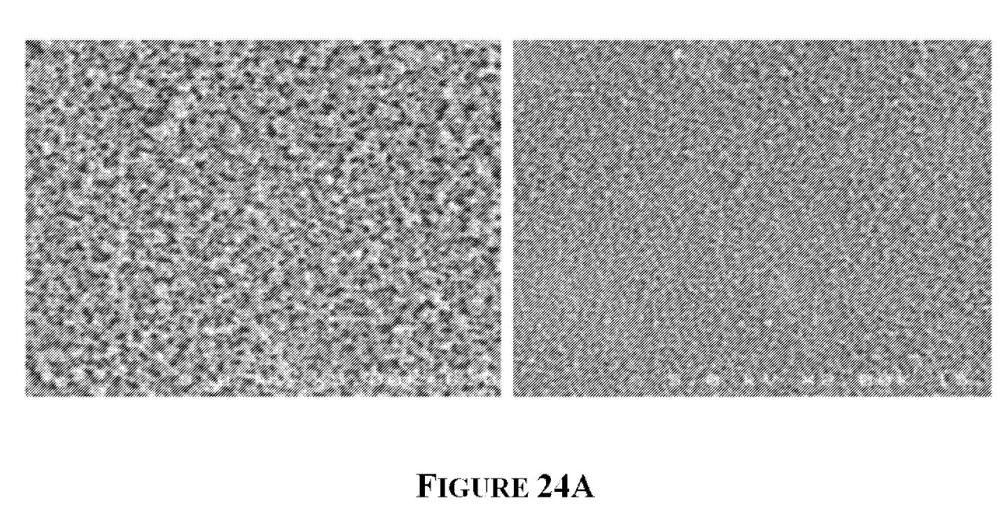

The first layer was CH (2 mg/ml; 10 min). The second layer was PSS (2 mg/ml) with 0.1% nanoparticle for 2 min. The resultant micrograph is shown in FIG. 20.

### m. EXAMPLE 13

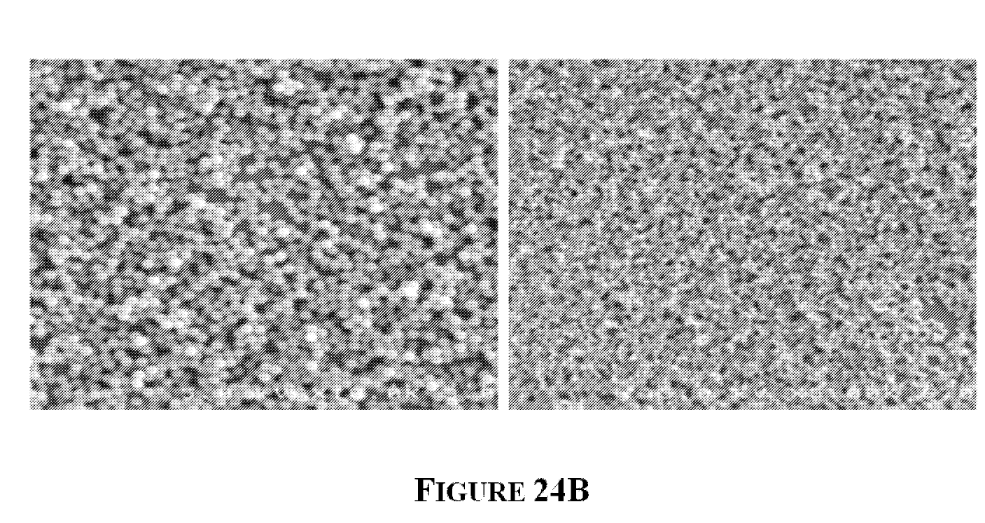

The first layer was CH (2 mg/ml; 10 min). The second layer was PSS (2 mg/ml) with 0.1% nanoparticle for 3 min. The resultant micrograph is shown in FIG. 21.

### n. EXAMPLE 14

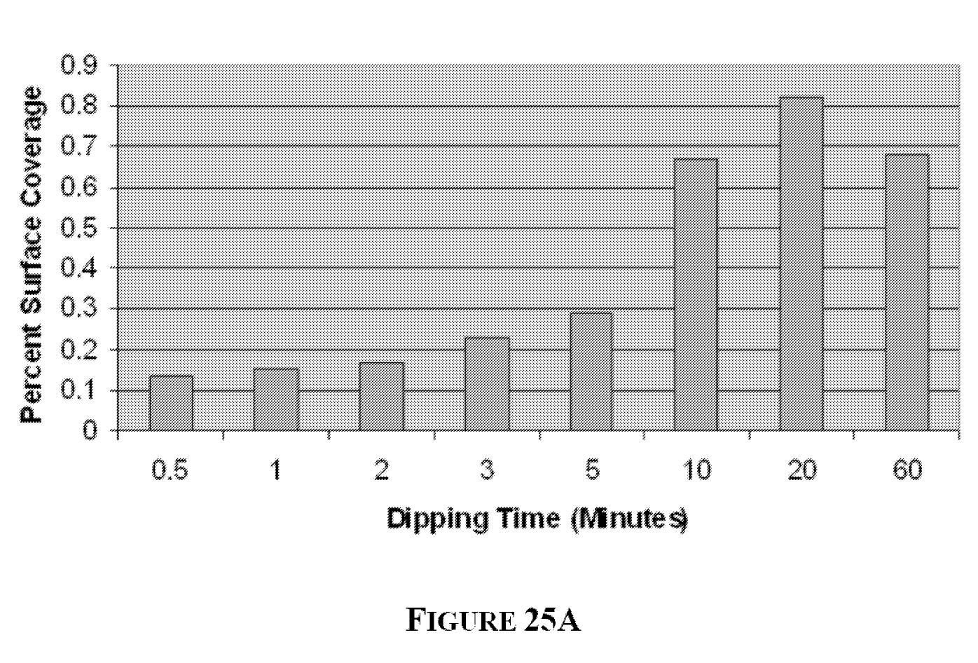

The first layer was CH (2 mg/ml; 10 min). The second layer was PSS (2 mg/ml) with 0.1% nanoparticle for 5 min. The resultant micrograph is shown in FIG. 22.

### o. EXAMPLE 15

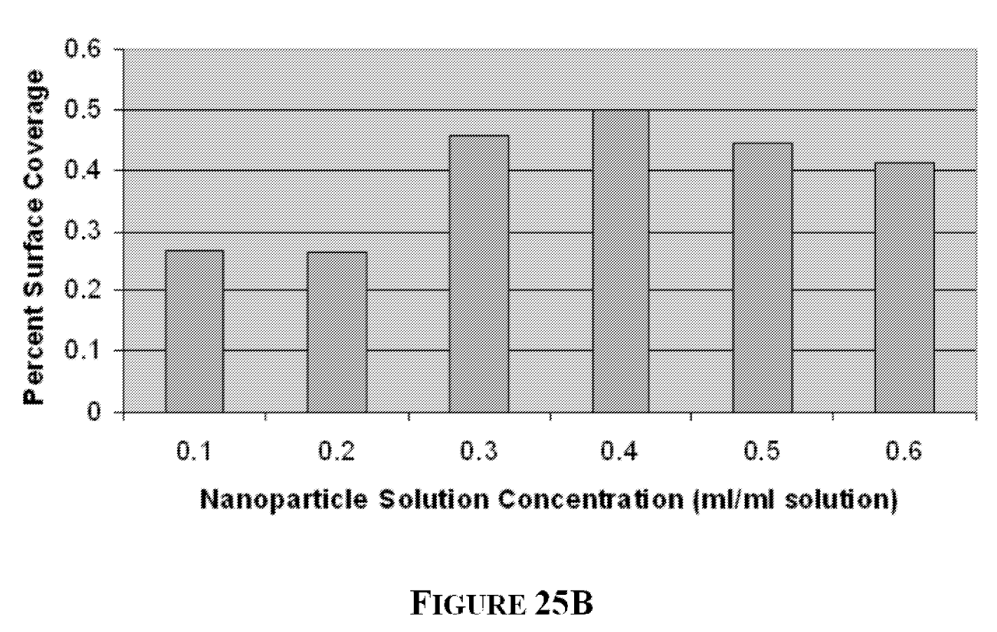

The first layer was CH (2 mg/ml; 10 min). The second layer was PSS (2 mg/ml) with 0.1% nanoparticle for 60 min. The resultant micrograph is shown in FIG. 23.

### p. EXAMPLE 16

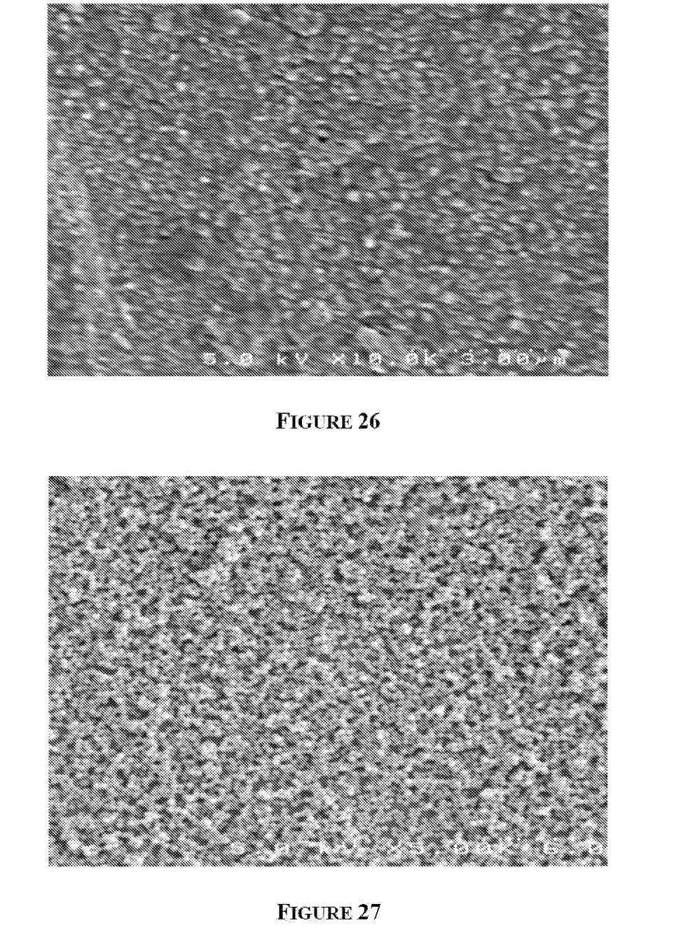

The first layer was CH (2 mg/ml) in 25% acetic acid with 0.14M NaCl solution for 20 minutes. The second layer 0.4% nanoparticle (NP) solution in PSS (2 mg/ml) for 10 min. Subsequent layers of PSS and CH for 10 min each (5 layers of each). The resultant micrographs are shown in FIG. 24.

### q. EXAMPLE 17

The deposition of layers and/or payloads (i.e., “chemical information”) was carried out using the layer-by-layer (LBL) polyelectrolyte assembly approach developed by Gero Decher (Science 1997, 277, 1232-1237.). A hybrid system composed of naturally occurring polysaccharides and polyionic polymers was used as building blocks. 316L stainless steels foils (Goodfellow Corporation), 316L stainless steel and cobalt chromium stents, generously provided by Guidant Corporation (Santa Clara, Calif.) were used as model substrates. A typical surface modification process involves first dipping the substrate in a polysaccharide (e.g., chitosan) solution followed by sequential, alternating incubation in solutions bearing oppositely charged species. This process was typically repeated 4 times to yield a base surface modification layer. Additionally, depending on the design consideration, some of the layers included gold nanoshells, to improve contrast in the CT mode and supramolecular complexes of Gadolinium for MR imaging. Gold nanoshells (5 nm) and polystyrene nanoparticles (PS-NP) (300 nm) were studied as model particulate systems for incorporation on stent surfaces. Incorporation of these moieties and gadolinium was achieved by co-adsorption in presence of the major polyelectrolyte. The modified substrates were imaged using a field emission scanning electron microscope (Hitachi S4200). For the surface coverage experiments, only PS-NP was employed due to its larger size. The surface area coverage was determined by analyzing the SEM images using Scion Image (NIHI), image analysis freeware, following which the system was optimized using an iterative approach.

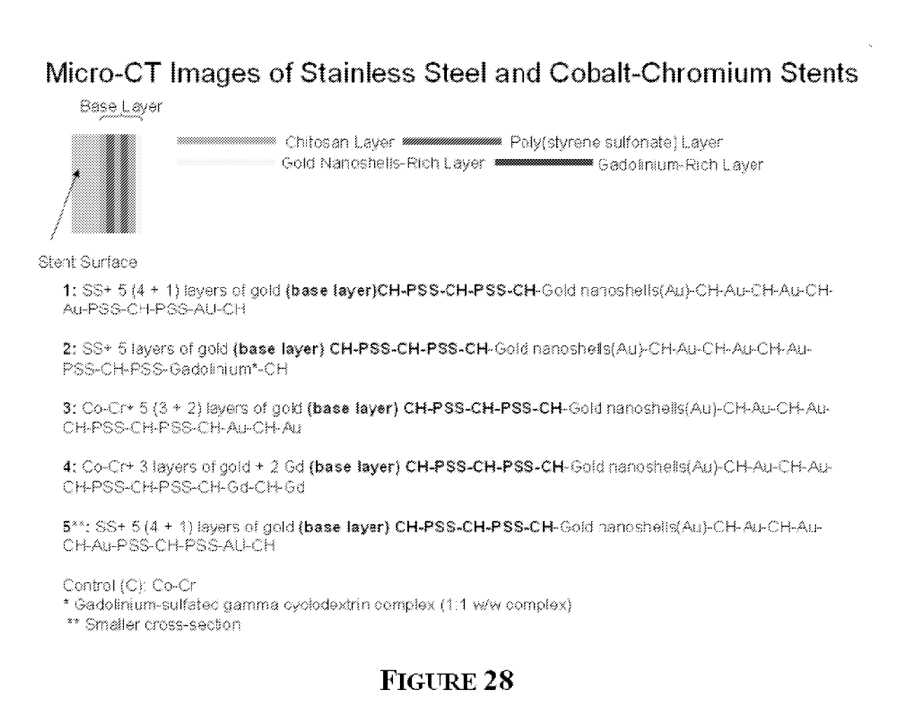

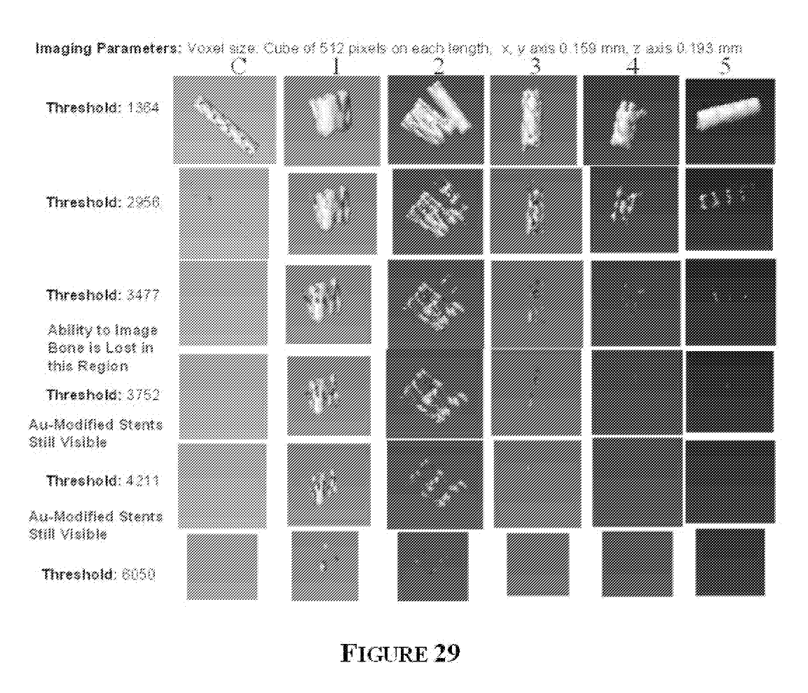

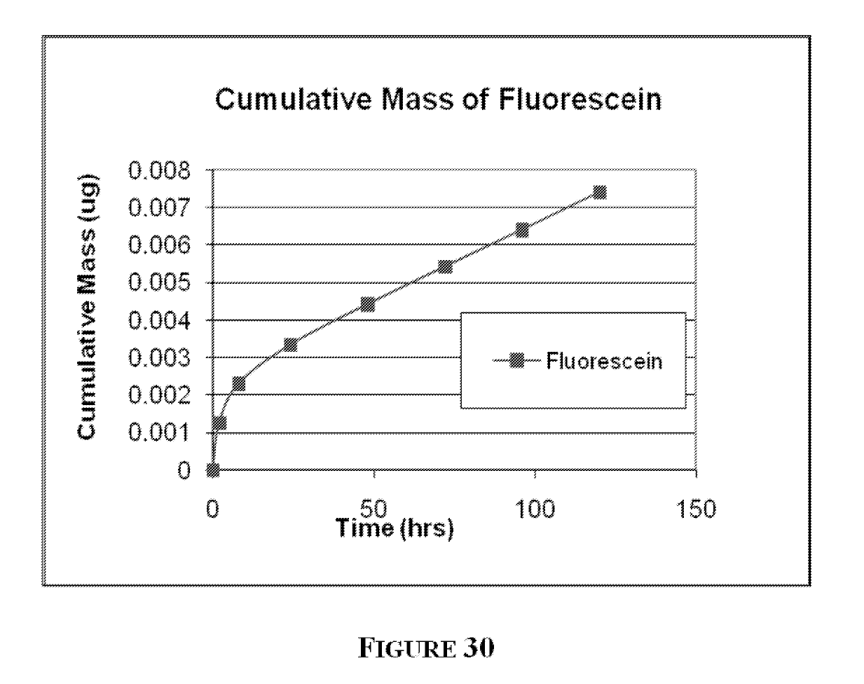

In the LBL approach, alternating layers composed of charged moieties of opposite charge characteristics were assembled on a surface from solution. The strength of this approach lies, at least in part, in its simplicity and ability to impart information defined at a thickness of few nanometers. By appropriate choice of the co-adsorbent, nanoparticle of differencing charge and chemical characteristics can be co-adsorbed and, hence, deposited onto a stent surface. FIG. 25 shows the relationship between incubation (dipping) time and PS-NP surface coverage. FIG. 26 shows a sample surface coated with gold nanoparticles with nearly 100% surface coverage. While surface deposition of small gold nanoshells is interesting, one strength of the approach lies in the ability to modify a material surface with large moieties, such as the 200 nm PS-NP, with near complete surface coverage (FIG. 27). These nanoparticle-modified stents are can be evaluated using CT and MR modalities.

### r. EXAMPLE 18

Nanoparticles comprised of poly(lactic-co-glycolic) acid (PLGA) were prepared by adding 1 mL of an aqueous phase to an equal volume of a polymer dissolved in a THF/acetone binary solvent system. The PLGA concentration was from about 10 to about 20 mg/mL. To functionalize the nanoparticle surface, the aqueous phase was supplemented with either a PEG at 0.5 w/v %. The resulting nanoparticle suspension had a blue tint and was purified by dialysis to remove organic components and any un-trapped water soluble species. The resulting suspension was then further concentrated by dialysis to yield a stable suspension of about 2 w/v %.

### s. EXAMPLE 19

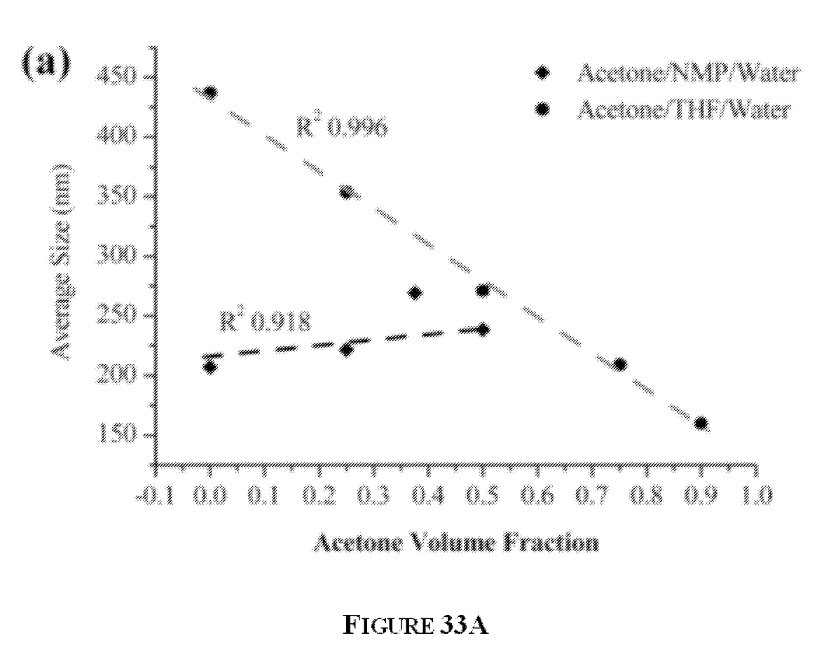

A PLGA nanoparticle comprising fluorescein diacetate was prepared, wherein the nanoparticle surface was functionalized with a poly(styrene sulfonate) (PSS). The resulting nanoparticles were rendered on a stainless steel foil surface by electrostatic assembly using the LBL methodology. The nanoparticles were suspended at a concentration of 0.3 w/v % in Chitosan (CH) solution and then assembled using alternating layers of CH and PSS. FIG. 30 shows the release profile of fluorescein diacetate from the stainless steel foil surface modified with PLGA nanoparticles comprising fluorescein diacetate.

### t. EXAMPLE 20

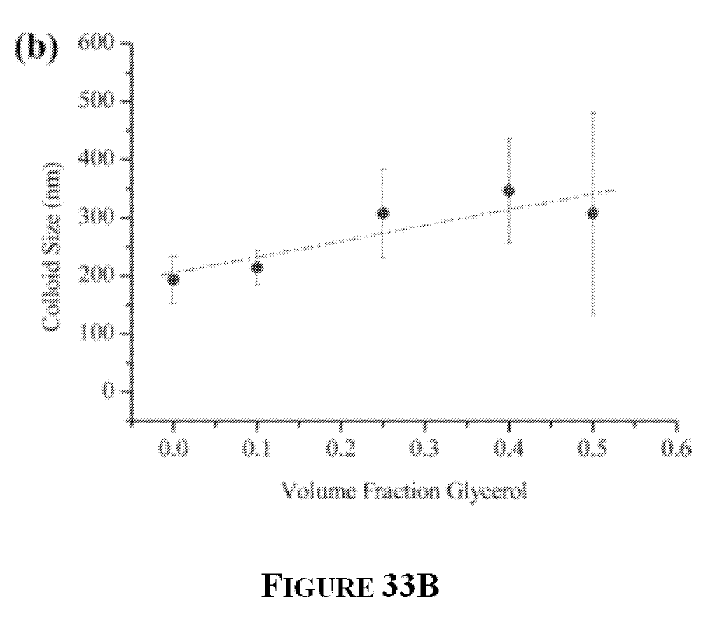

A PSS functionalized PLGA nanoparticle was prepared containing bovine serum albumin (BSA). A separate PSS functionalized PLGA nanoparticle was prepared containing horseradish peroxidase (HRP). FIG. 31 shows release profiles for BSA from two stainless steel foil surfaces. The first stainless steel foil surface (represented by diamonds) was modified with only BSA containing nanoparticles. The second stainless steel foil surface (represented by squares) was modified with both nanoparticles modified with BSA and nanoparticles modified with HRP.

### u. EXAMPLE 21

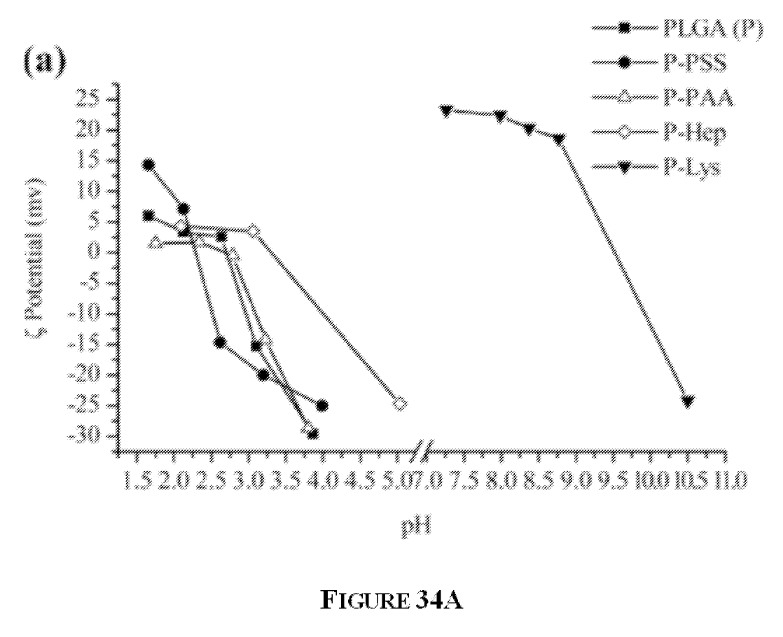

A PSS functionalized PLGA nanoparticle was prepared containing HRP. PSS functionalized PLGA nanoparticles were also prepared that separately contained BSA and fluorescein (FLR). FIG. 32 shows release profiles for HRP for a stainless steel surface modified with only nanoparticles containing HRP (represented by diamonds); a stainless steel surface modified with both nanoparticles containing HRP and nanoparticles containing BSA (represented by squares); and a stainless steel surface modified with both nanoparticles containing HRP and nanoparticles containing FLR.

### v. EXAMPLE 22

### Preparation of Nanoparticles

Materials. Poly(DL-lactide-co-glycolide) (PLGA, RG 503, MW=30,000) and poly(L-lactide) (PLA) MW=70,000, inherent viscosity 1.20 dL/g in CHCl3) were purchased from Birmingham Polymers (Birmingham, Ala., USA) and were purified by precipitation from methylene chloride in methanol prior to use. Tetrahydrofuran (THF), acetone (Ac), and 1-methyl-2-pyrolidone (NMP) were purchased from either Aldrich (Sigma-Aldrich, Milwaukee, Wis., USA) or Fisher (Fisher Scientific, Pittsburgh, Pa., USA) and used as received. All solvents were HPLC grade or the highest available purity. Poly(styrene sulfonate) (PSS, MW=70,000), poly(acrylic acid) (PAA, MW=2,000), poly(L-lysine hydrochloride) (PLys, MW=22,100), poly(ethylene glycol) (PEG, MW=10,000), and porcine heparin were purchased from Sigma and used as received without further purification. Doubly distilled deionized (DI) water obtained from a Milli-Q water purification system (Millipore, Bedford, Mass.) was used throughout the study.

Preparation of Nanoparticles. To prepare nanoparticles, 1 mL of the aqueous phase was added to an equal volume of polymer dissolved in a binary solvent system. The typical polymer concentration was 10 or 20 mg/mL. The solvent pairs used in this study include THF/acetone and NMP/acetone. The volumetric ratio of the solvent pair was optimized to yield nanoparticles of various sizes. When surface functionalization was desired, the aqueous phase was supplemented with either a polyelectrolyte or a water-soluble polymer such as PEG at 0.05 w/v %. The resulting nanoparticle suspension had a blue tint (Tyndall effect), was purified by dialysis to remove organic components and any untrapped water-soluble species, and was concentrated further by dialysis to yield stable suspensions of about 2.0 w/v %.

Determination of Nanoparticle Size and Zeta Potential. Nanoparticle size and zeta potential were determined using a Malvern Zetasizer (3000HS, Malvern Instruments Ltd., Malvern, U.K.). All measurements were made in automatic mode, and the software supplied by the manufacturer was used to analyze the data. For size measurements, the nanoparticle suspension was diluted by a factor of 15 with DI water prior to analysis, and for zeta measurements, the pH of the nanoparticle suspension was adjusted to the desired pH using either HCl or NaOH prior to analysis. XPS Surface Analysis. For XPS analysis, 5 mL of the nanoparticle suspension in water was dialyzed against 500 mL of 50% ethanol, flash frozen in liquid nitrogen, and then lyophilized for 48 h to a powder. The nanoparticle powder was then placed on the sample stub, and a Kratos Axis-Ultra X-ray photoelectron spectrometer equipped with a monochromatic Al K\acute{α} (1486 eV) X-ray source operating at 315 W (25 mA) was used to collect XPS data. High-resolution data was collected using a pass energy of 40 eV in 0.05 eV steps. The elemental composition was calculated, and curve-fitting routines were performed with CasaXPS software. Mass fraction of the functionalizing agents on the nanoparticle surface was determined by comparing the XPS spectra of functionalized nanoparticle with that of pure PLGA and functionalizing agent using CasaXPS software routine.

Florescence Microscopy. DAPI, a water-soluble negatively charge fluorescent dye, was used to visualize the nanoparticles. Nanoparticles containing DAPI were prepared by adding DAPI (Vecta Shield, Vector Laboratories, CA; solution in glycerol) to the aqueous phase prior to nanoaprticle formation (3 drops in 5 mL of water). The nanoparticle suspension was dialyzed against deionized water for 48 h to remove free DAPI and then photographed using a Zeiss Axiophot fluorescence microscope at 400× magnification.

Effect of Organic- and Aqueous-Phase Composition on Nanoparticle Size. Drago's solvent polarity index was utilized to select the binary solvent system that was capable of dissolving biodegradable polymer P(DL)LGA, one of the most commonly used polymers in injectable sustained release systems, at high concentrations (1-4 w/v %) while still exhibiting miscibility with water. Using this scale, we identified two solvent pairs that satisfied these requirements, namely, tetrahydrofuran/acetone (THF/Ac) (Sys I) and N-methylpyrrolidone/acetone (NMP/Ac) (Sys II) (polarity: water≈NMP>Ac>THF). The choice of these specific solvent pairs would also enable the verification of the role of water in nanoparticle formation, which is central to the hypothesis. In Sys I, increasing the volume fraction of acetone resulted in nanoparticles of smaller mean diameter, whereas in Sys II, increasing nanoparticles of increasing mean diameter (FIG. 33a). These results are consistent with what one would expect on the basis of the miscibility of the system with water because increased miscibility due to increased polarity (i.e., Sys I) should promote more rapid polymer-phase gelation (extended coil to collapsed-coil transition) and decreased miscibility due to lower polarity (i.e., Sys II) should slow down the kinetics of this gelation process (FIG. 33a). All solvent systems yielding nanoparticles will narrow the polydispersity index ranging from 0.05 to 0.09. The effect of the aqueous-phase viscosity on nanoparticle size was also studied. The viscosity of the aqueous phase was modulated through the addition of glycerol, and its impact on nanoparticle size was studied (FIG. 33b). A linear correlation between higher solution viscosity and increased nanoparticle was observed. More importantly, however, greater variability in nanoparticle size was observed upon increasing glycerol concentration. This is an expected outcome because increased aqueous-phase viscosity would impair water diffusion into the organic phase, making the sol-gel transition in the polymer phase less sharp.

Functionalization of the PLGA Nanoparticle Surface. Nanoparticles bearing various surface-bound functionalities such as PEG, heparin, poly(lysine), PSS, and PAA were prepared by incorporating the macromolecules bearing the functionality of choice (i.e., PEG, heparin, PSS, PAA, and PLys) at a low concentration of 0.05% during nanoparticle formation. The presence of the appropriate surface functionality was verified by measuring the isoelectric point (pIe) of the nanoparticle surface by mapping the zeta potential as a function of pH (FIG. 34a and Table 1, below). As seen in FIG. 34 and Table 1, pIe of the nanoparticle surface compared well with what would be expected on the basis of the ionizable moieties in the surface-bound functionality. A more quantitative, definitive verification was obtained by carrying out XPS analysis of the nanoparticle surface (Table 2, FIG. 35). XPS analysis revealed not only information about the surface of the nanoparticle but also that very high surface coverage of functional moieties was attainable in some cases. For example, negatively charged high-molecular-weight species such as PSS yielded an excess of 50% surface coverage (Table 2). While not wishing to be bound by theory, this suggests that the entrapment of functional groups on the nanoparticle surface during nanoparticle formation might be dictated by the molecular weight of the macromolecules bearing the functional groups. This is reasonable because an increase in the chain length of the macromolecule would improve entanglement and thus entrapment within the gelling polymer phase. A notable observation was that the morphology of the nanoparticle and the size were not significantly impacted by the introduction of a functionalization process (FIG. 34b).

TABLE 1
Nanoparticle (NP) Surface Characteristics:
Correlation between the Isoelectric Point (pIₑ) of the NP Surface
with the pKₐ of the Functional Group^{a}

| | pKₐ of | pIₑ of the | Δζ from | ζ at |
|---|---|---|---|---|
| NP composition | surface group | NP surface | PLGA | pH 7.4 |
| PLGA (P) | | 2.75 | 0 | −26.7 |
| P-PSS | ~2 | 2.40 | −0.35 | −28.3 |
| P-PAA | ~3.5 | 2.80 | +0.05 | −26.2 |
| P-PLys | ~10 | 9.50 | +6.75 | 16.9 |
| P-Hep | 2-4^{b} | 3.40 | +0.65 | −28.1 |
| P-PEG | | N/A^{c} | | −28.3 |

^{a}PSS, poly(styrene sulfonate); PAA, poly(acrylic acid); PLys, poly(L-lysine); Hep, heparin; PEG, poly(ethyleneoxide).
^{b}Estimated.
^{c}NP coagulated before pIₑ could be determined,

TABLE 2
C is Composition of the NP Surface

| | | | | | percent of |
|---|---|---|---|---|---|
| | | | | | surface mass |
| | | | | | contributed |
| | | | | | by functional |
| | COOR | COO—C—OR | C—O | C—Hₓ | groups |
| PLGA | 38.2 | 36.7 | nd | 25.1 | |
| std dev | 0.0 | 0.0 | | 0.0 | |
| PLGA-Plys | 27.1 | 28.0 | 9.0 | 36.0 | 24 |
| std dev | 3.1 | 3.2 | 1.6 | 4.7 | 9 |
| PLGA-PSS | 12.1 | 12.5 | nd | 75.5 | 66 |
| std dev | 1.6 | 1.6 | | 3.2 | 4 |
| PLGA-PAA | 36.9 | 35.5 | nd | 27.5 | 3 |
| std dev | 0.7 | 0.7 | | 1.4 | 2 |
| PLGA-heparin | 12.9 | 8.6 | 33.6 | 32.6 | 76 |
| std dev | 1.3 | 0.2 | 1.0 | 3.7 | 1 |
| PLGA-PEG | 27.6 | 26.5 | 13.5 | 32.4 | 28 |
| std dev | 1.3 | 4.1 | 1.5 | 9.8 | 11 |

It was found that PLGA nanoparticles could be prepared by the addition of water to both solvent-pair systems without the need for solvent evaporation or a hardening step. As described herein, in a typical process, rapid mixing of PLGA dissolved in an organic solvent pair with an equal volume of an aqueous phase resulted in the instantaneous formation of nanoparticles that, when concentrated, yielded stable suspensions at even 2 w/v %. It was observed that aging of the nanoparticles suspension did not result in an increase in nanoparticle size, suggesting that the solidification of the nanoparticle is rapid. The yields based on the initial polymer mass was >90%. It was also observed that with respect to nanoparticle size, increasing acetone volume fraction in Sys I decreased the nanoaprticle size (R²=0.996) whereas in Sys II it increased the NP size (R²=0.918) (FIG. 33a). This observation is consistent with a mechanism that involves the precipitation of the polymer (gelation) that is driven by the diffusion of water into the polymer salvation shell. In such a process, an increased rate of water diffusion should favor the faster transition of the polymer chains to a collapsed coil, yielding denser, smaller particles, whereas a diminution in water diffusion due to lower miscibility should slow down this process, resulting in larger particles. In fact, nanoparticles ranging in size from 70-500 nm were obtained without the need for any steric stabilization agents. It should be noted that the ability to tune nanoparticle size can be a factor in tumor targeting because nanoparticle size has been shown to be a factor in the accumulation of nanoparticles in tumor vasculature and within tumors through the passive “enhanced permeation retention” mechanism. The polydispersity index (PDI) of the nanoparticles was determined and was found to be quite narrow, with values of less than 0.1. Further evidence to support a mechanism of nanoparticle formation through the diffusion of water was obtained by studying the effect of aqueous-phase viscosity on nanoparticle size. Upon increasing the viscosity of water by the addition of glycerol, which is capable of hydrogen bonding with water and hence is miscible with water, an increase in nanoparticle size was observed (FIG. 33b). It was more pronounced at higher glycerol volume fractions and appears to be consistent with the slower diffusion of water with increased viscosity, resulting in a slower rate of nucleation and growth of nanoparticles. Stability studies have been conducted, indicating that all of the functionalized PLGA-nanoparticle suspensions are stable over at least a 3 month period, as ascertained by visual inspection for aggregates and sediments and light scattering (data not shown). It has been observed that once instability sets in rapid coagulation ensues and results in a translucent mass in the bottom of the test tube and a clear supernatant that does not have blue coloration. No such phase separation was observed in any of the samples over the 3 month period.

On the basis of the above mechanistic insight, the formation of nanoparticles using a water phase rich in synthetic polyions (0.05% w/v) was explored as a means of imparting functionality to the nanoparticle surface. It was observed that the introduction of polyions into the aqueous phase did not hinder the formation of nanoparticles and had a minimal impact on the size and polydispersity of the nanoparticles. Furthermore, we observed that nanoparticles produced under these conditions possessed surface charge characteristics consistent with the chemical structure of the polyion in solution as determined by zeta potential measurements (Table 1). Specifically, the surface charge in these nanoparticles exhibited a charge inversion close to the pKa of the ionizable group in the polyion (FIG. 34a). While not wishing to be bound by theory, this is consistent with a working hypothesis that surface functionality could be introduced into an nanoparticle via the entrapment of polyions from the water phase during nanoparticle formation. Using the approach described herein, nanoparticle bearing heparin, a naturally occurring anticoagulant, can be prepared under identical conditions (FIG. 34a) without the a priori need to synthesize PLGA polymers bearing the heparin moiety. The functionalization of polymers with sugars is very challenging because of the complexity of sugar chemistry. The presence of heparin and other functional polymers studied herein was verified using XPS. (XPS spectra of PLGA (without functionality), PEG, and heparin are shown, but XPS spectra of nanoparticles with PSS, PAA, and PLys are not shown.) The surface coverage of the functional polymers as determined by the contribution of the functional polymers to the mass of the nanoparticle surface ranged from 3 to >70% in the case of heparin (Table 2).

To determine if the polyionic species was incorporated into the nanoparticle structure via physical entrapment or surface adsorption, we studied the changes in the zeta potential of PLGA-PLys as a function of increasing ionic strength. The choice of PLGA-PLys for these studies was based on the rationale that because the unmodified PLGA surface has a negative zeta potential at pH 6 to 7 (pH range of the process) it is most likely to favor the electrostatic adsorption of polycations. Upon increasing the ionic strength of a PLGA-PLys nanoparticle suspension from 0.3 to 24 mM using potassium chloride, we observed that the surface charge characteristics were retained (zeta before =48 mV; zeta after =34 mV), suggesting that the moieties contributing to the surface characteristics of the nanoparticle are not desorbed and hence are not electrostatically bound to the nanoparticle surface.

The ternary system described herein is not limited to the preparation of nanoparticles with polyionic functionality but may be extended to include nonionic species such as poly(ethylene glycol) (PEG). Nanoparticles bearing even low-molecular-weight PEG (MW=10 kDa) can be easily prepared by incorporating PEG into the water phase as a stable suspension without a significant impact on nanoparticle size. The presence of PEG functionality on the nanoparticle surface has been verified using XPS, as shown in FIG. 35.

Because the intended application of nanoparticles is to deliver bioactive agents, the process is amenable to the encapsulation of small water-soluble molecules using fluorescent dyes as models. In addition to P(DL)LGA, which is an amorphous polymer, functionalized nanoparticles of poly(L-lactic acid), a highly crystalline biodegradable polymer with wide applications in drug delivery, have been prepared as well.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A coated substrate comprising:
a. a substrate having a surface,
b. a cationic polymer layer adjacent the surface of the substrate,
wherein the cationic polymer layer comprises at least one residue of a first compound having the structure: 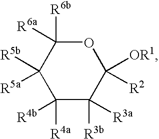
wherein R¹ is hydrogen or alkyl;
wherein R², R^{3a}, R^{3b}, R^{4a}, R^{4b}, R^{5a}, R^{5b}, R^{6a}, and R^{6b} are, independently, hydrogen, hydroxyl, alkyl, aryl, alkoxy, carboxyl, ester, amino, or amide, with the provisos that at least one of R², R^{3a}, R^{3b}, R^{4a}, R^{4b}, R^{5a}, R^{5b}, R^{6a}, and R^{6b} is amino and that at least one of R², R^{3a}, R^{3b}, R^{4a}, R^{4b}, R^{5a}, R^{5b}, R^{6a}, and R^{6b} is hydroxyl or alkoxy; and
c. an anionic polymer layer adjacent the cationic layer,
wherein the anionic polymer layer comprises at least one residue of a compound having the structure: 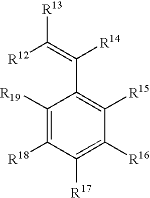
wherein R¹², R¹³, and R¹⁴ are, independently, hydrogen, alkyl, aryl, carboxyl, or ester; and
wherein R¹⁵, R¹⁶, R¹⁷, R¹⁸, and R¹⁹ are, independently, hydrogen, alkyl, aryl, alkoxy, amino, amide, carboxyl, or ester, with the proviso that at least one of R¹⁵, R¹⁶, R¹⁷, R¹⁸, and R¹⁹ is SO₃R¹¹, wherein R¹¹ is hydrogen or alkyl.

2. The coated substrate of claim 1, wherein the outermost polymer layer has a surface having fractal characteristics.

3. The coated substrate of claim 1, wherein the anionic polymer layer is positioned between the surface and the cationic polymer layer.

4. The coated substrate of claim 1, wherein the substrate is a stent, an artificial joint, an artificial organ, a bone screw, a bone plate, or a tissue.

5. The coated substrate of claim 1, wherein the substrate comprises a material selected from stainless steel, cobalt-chromium alloy, titanium, Nitinol, ceramic, and polymer.

6. The coated substrate of claim 1, wherein the anionic polymer layer comprises a polymer having the structure: 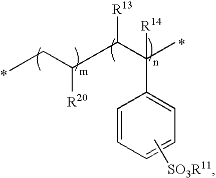
wherein R²⁰ is hydrogen, alkyl, or aryl;
wherein m is zero or a positive integer; and
wherein n is zero or a positive integer.

7. The coated substrate of claim 6, wherein the anionic polymer layer comprises one or more of polystyrene sulfonate, poly(acrylic acid), poly(methacrylic acid), substituted poly(phosphazene), poly(vinyl alcohol), heparin sulfate, chondroitin sulfate, dermatan sulfate, heparin, poly(aspartic acid), poly(tyrosine), copolymers of aspartic acid and tyrosine, other negatively charged poly amino acids, dextrans, or poly(glutamic acid), or blends or copolymers thereof.

8. The coated substrate of claim 1, wherein the cationic polymer layer comprises a polymer having the structure: 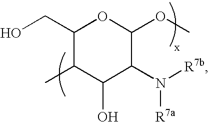
wherein R^{7a} and R^{7b} are independently hydrogen, alkyl, or acyl;
wherein x is a positive integer.

9. The coated substrate of claim 8, wherein the cationic polymer layer comprises poly-D-glucosamine.

10. The coated substrate of claim 1, wherein the cationic polymer layer further comprises at least one of chitosan, chitin, poly(L-lysine), poly(histidine), poly(imidazole), or poly(allylamines).

11. The coated substrate of claim 1, wherein the anionic polymer layer further comprises at least one of poly(styrene sulfonate), hyaluronic acid, alginate, or poly(glutamic acid).

12. The coated substrate of claim 1, wherein at least one polymer layer further comprises a payload comprising at least one imaging agent, at least one magnetically active agent, at least one pharmaceutically active agent, at least one biologically active agent, at least one functionalized polymeric nanoparticle, or at least one functionalized lipid nanoparticle.

13. A coated substrate comprising:
a. a substrate having a surface,
b. a cationic polymer layer adjacent the surface of the substrate,
c. an anionic polymer layer adjacent the cationic polymer layer, and
d. at least one nanoparticle or microparticle positioned within the anionic polymer layer.

14. The coated substrate of claim 13, wherein the cationic polymer layer comprises at least one residue of a first compound having the structure: 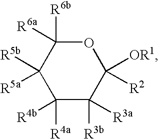
wherein R¹ is hydrogen or alkyl;
wherein R², R^{3a}, R^{3b}, R^{4a}, R^{4b}, R^{5a}, R^{5b}, R^{6a}, and R^{6b} are, independently, hydrogen, hydroxyl, alkyl, aryl, alkoxy, carboxyl, ester, amino, or amide, with the provisos that at least one of R², R^{3a}, R^{3b}, R^{4a}, R^{4b}, R^{5a}, R^{5b}, R^{6a}, and R^{6b} is amino and that at least one of R², R^{3a}, R^{3b}, R^{4a}, R^{4b}, R^{5a}, R^{5b}, R^{6a}, and R^{6b} is hydroxyl or alkoxy; and
wherein the anionic polymer layer comprises at least one residue of a compound having the structure: 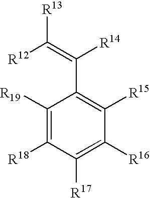
wherein R¹², R¹³, and R¹⁴ are, independently, hydrogen, alkyl, aryl, carboxyl, or ester; and
wherein R¹⁵, R¹⁶, R¹⁷, R¹⁸, and R¹⁹ are, independently, hydrogen, alkyl, aryl, alkoxy, amino, amide, carboxyl, or ester, with the proviso that at least one of R¹⁵, R¹⁶, R¹⁷, R¹⁸, and R¹⁹ is SO₃R¹¹, wherein R¹¹ is hydrogen or alkyl.

15. The coated substrate of claim 13, wherein the at least one nanoparticle or microparticle comprises at least one nanoparticle selected from a quantum dot, a gold nanoparticle, and a silicon nanoparticle.

16. A method of making a coated substrate comprising the steps of:
a. providing a substrate having a surface;
b. contacting the surface with an ionic polymer solution, thereby disposing an ionic polymer layer adjacent to the surface; and
c. contacting the ionic polymer layer with a counterionic polymer solution, thereby disposing a counterionic polymer layer adjacent to the ionic polymer layer.

17. The method of claim 16, wherein one of the ionic polymer layer and the counterionic polymer layer comprises at least one residue of a first compound having the structure: 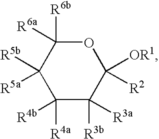
wherein R¹ is hydrogen or alkyl;
wherein R², R^{3a}, R^{3b}, R^{4a}, R^{4b}, R^{5a}, R^{5b}, R^{6a}, and R^{6b} are, independently, hydrogen, hydroxyl, alkyl, alkoxy, carboxyl, ester, amino, or amide, with the provisos that at least one of R², R^{3a}, R^{3b}, R^{4a}, R^{4b}, R^{5a}, R^{5b}, R^{6a}, and R^{6b} is amino and that at least one of R², R^{3a}, R^{3b}, R^{4a}, R^{4b}, R^{5a}, R^{5b}, R^{6a}, and R^{6b} is hydroxyl or alkoxy; and
wherein the other of the ionic polymer layer and the counterionic polymer layer comprises at least one residue of a compound having the structure: 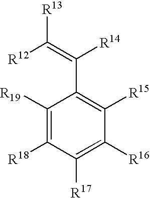
wherein R¹², R¹³, and R¹⁴ are, independently, hydrogen, alkyl, carboxyl, or ester; and
wherein R¹⁵, R¹⁶, R¹⁷, R¹⁸, and R¹⁹ are, independently, hydrogen, alkyl, alkoxy, amino, amide, carboxyl, or ester, with the proviso that at least one of R¹⁵, R¹⁶, R¹⁷, R¹⁸, and R¹⁹ is SO₃R¹¹, wherein R¹¹ is hydrogen or alkyl.

18. The method of claim 17, wherein the anionic polymer layer comprises a polymer having the structure: 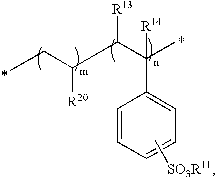
wherein R²⁰ is hydrogen, alkyl, or aryl;
wherein m is zero or a positive integer; and
wherein n is zero or a positive integer.

19. The method of claim 17, wherein the cationic polymer layer comprises a polymer having the structure: 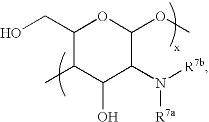
wherein R^{7a} and R^{7b} are independently hydrogen, alkyl, or acyl;
wherein x is a positive integer.

20. The method of claim 17, wherein one or both of the ionic polymer solution and the counterionic polymer solution further comprises least one nanoparticle or microparticle.

21. The method of claim 20, wherein the anionic polymer solution further comprises least one nanoparticle or microparticle.

22. A method of treating comprising the step of implanting the coated substrate of claim 1 into a subject.

23. The method of claim 22, wherein at least one polymer layer further comprises a payload comprising at least one imaging agent, the method further comprising the step of imaging the coated substrate.

24. A method of performing radio frequency ablation comprising the steps of:
a. providing the coated substrate of claim 1, wherein the coated substrate or the product further comprises at least one metal nanoparticle or metal microparticle; and
b. exposing the coated substrate or the product to radio frequency radiation.

25. The method of claim 24, wherein the metal nanoparticle or the metal microparticle comprises gold.
